(19) United States
(12) Patent Application Publication
(10) Pub. No.: US 2020/0354664 A1
(43) Pub. Date: Nov. 12, 2020

(54) A Bioactive 3D Encapsulation Culture System For Cell Expansion

(71) Applicant: Rutgers, The State University of New Jersey, New Brunswick, NJ (US)

(72) Inventors: Biju Parekkadan, Piscataway, NJ (US); Ayesha Aijaz, Piscataway (US)

(21) Appl. No.: 16/760,393

(22) PCT Filed: Nov. 21, 2018
(86) PCT No.: PCT/US2018/062229

§ 371 Date: Apr. 29, 2020

Related U.S. Application Data
(60) Provisional application No. 62/589,934, filed on Nov. 22, 2017.

(51) Int. Cl. C12M 1/12; C12N 5/00; C12N 5/0775

(52) U.S. Cl. CPC C12M 25/16; C12N 2533/30; C12N 5/0663; C12N 5/0012

(57) ABSTRACT

Systems and methods for growing cells are provided. A capsule for growing or storing cells includes a shell defining an interior compartment and a substrate for cell attachment located within the compartment. The substrate comprises a polymer and one or more adhesion molecules. The substrate for cell attachment can be an inner surface of the shell and/or a hydrogel disposed within the interior compartment. The capsule can further include a cell, such as a stem cell, adhered to the substrate

### RELATED APPLICATION

This application claims the benefit of U.S. Provisional Application No. 62/589,934, filed on Nov. 22, 2017. The entire teachings of the above application are incorporated herein by reference.

### BACKGROUND

Cells are found in many industrial applications ranging from production of vaccines to use as cell therapies. Suspension of cells in bioreactor systems has allowed for the automation and scale up of cell cultures for such industrial applications. Of particular interest and challenge, anchorage-dependent cells have been cultured as monolayers on two-dimensional (2D) tissue-culture treated surfaces, such as T-flasks, multilayer cell factories [3, 4], and roller bottles or hollow-fiber based bioreactor systems [5, 6]. More recently, a transition to culturing anchorage-dependent cells in suspension cultures has emerged, as suspension cultures have the capability of providing high yields in a more spatially efficient format than traditional 2D cultures, which require logistically impractical planar growth surface areas, particularly for commercial production scales.

Microcarriers, most commonly made of polystyrene and coated with collagen or laminin for cell attachment, have enabled suspension culture of anchorage-dependent cells in stirred-tank bioreactors [7-10]. Mesenchymal stem cells (MSCs) adhere to microcarriers and are cultured in stir tank bioreactors that range from 300 mL to 1000 L for large scale expansion [11]. Microcarrier-based bioreactor technology provides significant advantages, such as large surface area to volume ratio, process control, closed loop sampling and homogeneous culture conditions [12-16]. Despite the advantages of microcarrier technology, there remain a number of challenges to overcome, and there exists a need for improved systems and methods for culturing anchorage-dependent cells.

### SUMMARY

Methods and systems of the present invention provide for encapsulated cell culture systems that can be used for expansion and/or storage of cells.

In one embodiment, the invention relates to a capsule for growing or storing cells, such as adherent cells. The capsule includes a shell defining an interior compartment and a substrate for cell attachment located within the interior compartment. The substrate comprises a polymer that may contain one or more adhesion molecules.

In another embodiment, the invention relates to a method of storing cells that includes encapsulating cells in a capsule having a shell defining an interior compartment and a substrate for cell attachment located within the interior compartment. The cells adhere to the substrate within the interior compartment of the capsule.

In some embodiments, the substrate for cell attachment can be an inner surface of the shell and/or a hydrogel disposed within the interior compartment. The hydrogel can comprise cross-linked polyethylene glycol (PEG), cross-linked polyethylene glycol diacrylate (PEGDA), or a combination thereof. The shell can comprise a polymer, such as polyethylene glycol (PEG), polyethylene glycol diacrylate (PEGDA), alginate, chitosan, PEG copolymerized with alginate or chitosan, and PEGDA copolymerized with alginate or chitosan, or a combination thereof. Other polymers can include poly(lactic-co-glycolic acid) (PLGA), poly-L-lysine (PLL), polydimethylsiloxane (PDMS), polyacrylamide poly(N-isopropylacrylamide) (PNIPAAm), poly[2-(methyacryloxy)ethyl phosphorylcholine]-block-(glycerol monomethacrylate) (PMPC-PGMA), x-acetylene-poly(tert-butyl acrylate) (PtBA), poly(vinyl alcohol) (PVA), poly(acrylic acid) (PAA), poly(divinylbenzene-co-glycidylamethacrylate) (P(DVB-GMA)), poly(amidoamine) (PAMAM), poly(D-glucosamidoethylenemethacrylate) (PGAMA), poly(2-lactobionamido ethylmethacrylate (PLAMA), alkyl thioether end-functionalized poly(methacrylic acid) (PMAA-DDT), poly(ethylene glycol)-phosphine (PEG-phosphine), poly(vinyl pyrollidone) (PVP), poly(acrylic acid)-octylamine (PAA-octylamine), poly(maleic anhydride-alt-1-octadecene-block-poly(ethylene glycol) (PMAO-PEG). Additional examples of polymers can be found at Ladj R. et al., Polymer encapsulation of inorganic nanoparticles for biomedical applications, Intl. J. of Pharm., 2013. 458(1): p. 230-241, the entire contents of which is incorporated herein by reference.

In further embodiments, the adhesion molecule of the substrate can be an adhesion molecule that affects cell attachment to the material, cell viability, cell proliferation, cell survival, growth, and/or differentiation of the cell. For example, the adhesion molecule can be an adhesion peptide, such as RGDS (SEQ ID NO:1), YIGSR (SEQ ID NO:2), IKVAV (SEQ ID NO:3), REDV (SEQ ID NO:4), GKKQRFRHRNRKG (SEQ ID NO:5), RNIAEIIKDI (SEQ ID NO:6), KTRWYSMKKTTMKIIPFNR (SEQ ID NO:7), or any combination thereof. In addition, or alternatively, the adhesion molecule can be a partial or full-length protein, such as, for example, collagen type I, fibronectin, laminin, denatured collagen (also known as gelatin), collagen type IV, Matrigel® (Corning Life Sciences, Bedford, Mass.), poly-L-lysine (PLL), poly-D-lysine (PDL), or any combination thereof. Antibodies that engage with cell surface receptors (e.g., CD3 and CD28) and/or small molecules (e.g., nonpeptide small molecules such as stemregulin or reversine), for example, small molecules that activate a differentiation program in cells to enhance adhesion, can also be an adhesion molecule included on the substrate for cell attachment.

In further embodiments, the substrate further includes a growth factor, which can be located in the interior compartment, such as conjugated to the polymer, embedded within a hydrogel, and/or located within a liquid (e.g., a culture medium). The growth factor can be, for example, FGF, TGF-β1, VEGF, PDGF-BB, PDGF, IGF1, stem cell factor (SCF), thrombopoeitin (TPO), FMS-like tyrosine kinase 3 ligand (Flt-3L), erythropoietin, DL-1 notch ligand, Wnt, stromal derived factor (SDF)-1, interleukin (IL)-2, IL-3, IL-4, IL-6, IL-7, IL-15, IL-15R, CD40L, G-CSF, GM-CSF, 4-1BB and BMP superfamily members, or any combination thereof.

In yet further embodiments, the shell of the capsule is porous, having a pore size, for example, of about 10 nm to about 35 nm, or of about 20 nm. The shell can further include an enzyme-sensitive peptide, such as a protease-sensitive peptide, or other dissolvable material and conjugation moiety.

In yet further embodiments, the capsule includes a DNA-containing or RNA-containing molecule. The DNA-containing or RNA-containing molecule can be located in the interior compartment, such as conjugated to a polymer, embedded within a hydrogel, and/or located within a liquid (e.g., a culture medium) contained within the shell. The DNA or RNA-containing molecules can be, for example, cDNA, plasmid DNA, transposable DNA (e.g., using sleeping beauty transposons), viral vectors (e.g., adeno-associated virus, retrovirus, lentivirus, Sendai virus), modified RNA, siRNA, miRNA, antisense oligonucleotides, gene editing molecules, such as CRISPR/gRNA, zinc finger nucleases (e.g., TALENs), and meganucleases, as well as lipid vesicles containing these molecules, or any combination thereof.

In some embodiments, the capsule includes a cell adhered to a substrate, such as a stem cell. The cell can be, for example, a Mesenchymal Stem Cell (MSC), a Chinese Hamster Ovary (CHO) cell, a Madin-Darby Canine Kidney Epithelial (MDCK) cell, a Vero cell, a pancreatic islet, a peripheral blood mononuclear cell, an endothelial progenitor cell, a blood fibrocyte, a bone marrow cell, a T cell, a B cell, a dendritic cell, a CD34+ cell, an NK cell, a monocytes, a hepatocyte, a neural stem cell, a gastrointestinal cell, a skin cell, a skin cell progenitor cell, a cancer cell, a hybridoma cell, a prokaryotic cell, a HEK293T packaging cell line, a yeast cell, a pancreatic precursor cell, an embryonic stem cell, or an induced pluripotent stem cell. The cells may be encapsulated by exposing a porous shell of the capsule to the cells, the cells translocating through the pores into the interior compartment of the shell. Alternatively, the cells may be encapsulated during polymerization of the capsule. The capsule can include a culture medium in the interior compartment, thereby producing a suspension culture of encapsulated cells. The cells can grow and/or expand in a suspension culture. The suspension culture can be a stirred-tank suspension culture. Upon completion of a cell culturing or storage process, the shell of the capsule can be degraded and the cells harvested.

In another embodiment, the invention relates to a cell culture kit that includes first and second compositions. The first composition comprises a polymer precursor material and an adhesion peptide. The second composition comprises reagents for polymerizing the polymer precursor material to form a capsule having a shell that comprises a polymer produced by polymerization of the precursor material and the adhesion peptide. The adhesion peptide is present on an inner surface of the shell. The first composition can, optionally, further include a growth factor and/or a protease-sensitive peptide. The second composition can, optionally, further include a cross-linking reagent for forming a hydrogel within the interior compartment of the shell. The first composition may be lyophilized.

In another embodiment, the invention relates to a system for producing encapsulated cells that includes first and second compositions. The system comprises of a pumping apparatus, tubing sets, a specified light source, and liquid handling/collections bags. The first composition comprises a polymer precursor material and one or more substrates conjugated to the polymer. The second composition comprises reagents for polymerizing the polymer precursor material to form a capsule having a shell that comprises a polymer produced by polymerization of the precursor material and the conjugated substrate(s). The substrate(s) is present on an inner surface of the shell. The first composition can, optionally, further include a growth factor, DNA/RNA containing structure, and/or a protease-sensitive peptide. The second composition can, optionally, further include a cross-linking reagent for forming a hydrogel within the interior compartment of the shell. The first composition may be lyophilized.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram of a prior art microcarrier, shown in cross-section.

FIG. 2A is a diagram of an example of a capsule of the present invention in which cells adhere along an inner surface of the shell of the capsule.

FIG. 2B is a diagram of another example of a capsule of the present invention in which a shell includes a protease-sensitive peptide.

FIG. 2C is a diagram of an example of a capsule of the present invention in which cells are embedded in or adhered to a hydrogel core of the capsule.

FIG. 2D is a diagram of another example of a capsule of the present invention in which a hydrogel core includes covalently-bound growth factor (GF).

FIG. 2E is a diagram of yet another example of a capsule of the present invention in which a hydrogel core includes soluble growth factor (GF).

FIG. 2F is a diagram of another example of a capsule of the present invention in which the capsule includes an adhesion peptide, a protease-sensitive peptide, a growth factor (GF) and cells bound to a polyethylene glycol (PEG) chain.

FIG. 3A shows synthesized polyethylene glycol diacrylate (PEGDA) capsules cultured under static conditions (0 rpm).

FIG. 3B shows synthesized polyethylene glycol diacrylate (PEGDA) capsules cultured under agitation at a conventional speed (60 rpm).

FIG. 3C shows synthesized polyethylene glycol diacrylate (PEGDA) capsules cultured under agitation at a high speed (250 rpm).

FIG. 4A shows mesenchymal stem cells (MSCs) encapsulated in synthesized PEGDA microcarriers. Live cells (green, thick arrow) and dead cells (red, thin arrow) are shown, with a 200 μm scale.

FIG. 4B is a graph of cell number versus time in culture for two batches of encapsulated cells, each batch having a different input cell concentration.

FIG. 5 is a graph of absorbance versus glycine concentration for the detection of unreacted free amines in assessing conjugation efficiency of the adhesion peptide RGDS.

FIG. 6 shows rhodamine phalloidin (red) and DAPI (blue) stained encapsulated MSCs sampled on day 3 of a dynamic spinner flask culture.

FIG. 7 is graph of cell number versus time in culture for capsules having either cell-laden liquid cores (LC) or polymerized cores (PC) assessed under static cultures.

FIG. 8A shows live cells (green) and dead cells (red) in cell-laden polymerized core capsules sampled at day 1 of cell culturing at a high agitation speed (125 rpm).

FIG. 8B shows the capsules of FIG. 8A sampled at day 6.

FIG. 8C shows the capsules of FIGS. 8A and 8B sampled at day 10.

FIG. 8D is a graph of cell number versus time in days for the capsules of FIGS. 8A-8C.

FIG. 9A shows live cells (green) and dead cells (red) in cell-laden polymerized core capsules sampled at day 1 of cell culturing at a low agitation speed (30 rpm).

FIG. 9B shows the capsules of FIG. 9A sampled at day 3.

FIG. 9C shows the capsules of FIGS. 9A and 9B sampled at day 6.

FIG. 9D is a graph of cell number versus time in days for the capsules of FIGS. 9A-9C.

FIG. 10A is graph of the number of polymerized core (PC) capsules versus diameter of the capsules.

FIG. 10B is a graph of the surface area of the PC capsules of FIG. 10A versus diameter of the capsules.

FIG. 10C is a graph of the volume of the PC capsules of FIGS. 10A-10B versus diameter of the capsules.

FIG. 11A is graph of the number of liquid core (LC) capsules versus diameter of the capsules.

FIG. 11B is a graph of the surface area of the LC capsules of FIG. 11A versus diameter of the capsules.

FIG. 11C is a graph of the volume of the PC capsules of FIGS. 11A-11B versus diameter of the capsules.

FIG. 12A shows degradable capsules prior to exposure to a collagenase enzyme.

FIG. 12B shows the degradable capsules of FIG. 12A at three hours after exposure to collagenase.

FIG. 12C shows the degradable capsules of FIGS. 12A and 12B after exposure to collagenase overnight.

FIG. 12D shows the degradation of the capsules of FIGS. 12A-12C.

FIG. 13A shows cell-laden degradable capsules prior to exposure to a collagenase enzyme.

FIG. 13B shows the degradation of the capsules of FIG. 13A after mechanical agitation via pipetting.

FIG. 14A shows rhodamine phalloidin and DAPI stained encapsulated MSCs with a high adhesive concentration sampled on day 1.

FIG. 14B shows rhodamine phalloidin and DAPI stained encapsulated MSCs with a high adhesive concentration sampled on day 7.

FIG. 14C shows rhodamine phalloidin and DAPI stained encapsulated MSCs with a high adhesive concentration sampled on day 14.

FIG. 14D shows rhodamine phalloidin and DAPI stained encapsulated MSCs with a low adhesive concentration sampled on day 1.

FIG. 14E shows rhodamine phalloidin and DAPI stained encapsulated MSCs with a low adhesive concentration sampled on day 7.

FIG. 14F shows rhodamine phalloidin and DAPI stained encapsulated MSCs with a low adhesive concentration sampled on day 14.

FIG. 14G shows rhodamine phalloidin and DAPI stained encapsulated MSCs without adhesive sampled on day 1.

FIG. 14H shows rhodamine phalloidin and DAPI stained encapsulated MSCs without adhesive sampled on day 7.

FIG. 14I shows rhodamine phalloidin and DAPI stained encapsulated MSCs without adhesive sampled on day 14.

FIG. 15A shows calcein acetoxymethyl and ethidium homodimer1 stained MSCs in capsules with 0 mM PEG-RGDS sampled at day 1 of cell culturing.

FIG. 15B shows calcein acetoxymethyl and ethidium homodimer1 stained MSCs in capsules with 5 mM PEG-RGDS sampled at day 1 of cell culturing.

FIG. 15C shows calcein acetoxymethyl and ethidium homodimer1 stained MSCs in capsules with 10 mM PEG-RGDS sampled at day 1 of cell culturing.

FIG. 15D shows calcein acetoxymethyl and ethidium homodimer1 stained MSCs in capsules with 0 mM PEG-RGDS and PEG-FGF sampled at day 1 of cell culturing.

FIG. 15E shows calcein acetoxymethyl and ethidium homodimer1 stained MSCs in capsules with 5 mM PEG-RGDS and PEG-FGF sampled at day 1 of cell culturing.

FIG. 15F shows calcein acetoxymethyl and ethidium homodimer1 stained MSCs in capsules with 10 mM PEG-RGDS and PEG-FGF sampled at day 1 of cell culturing.

FIG. 16A shows calcein acetoxymethyl and ethidium homodimer1 stained cells following harvesting from capsules with 0 mM PEG-RGDS.

FIG. 16B shows calcein acetoxymethyl and ethidium homodimer1 stained cells following harvesting from capsules with 5 mM PEG-RGDS.

FIG. 16C shows calcein acetoxymethyl and ethidium homodimer1 stained cells following harvesting from capsules with 10 mM PEG-RGDS.

FIG. 17A shows conventional microcarriers one hour after equilibration in cell culture media.

FIG. 17B shows the microcarriers of FIG. 17A at 24 hours after incubation with MSCs under static conditions.

FIG. 17C shows the microcarriers and MSCs of FIG. 17B at 48 hours after dynamic culturing in a spinner flask at 70 rpm.

FIG. 18 is chart of cell viability under high agitation culture.

FIG. 19A shows MSCs encapsulated in synthesized PEGDA capsules without conjugated RGDS sampled at day 1.

FIG. 19B shows MSCs encapsulated in synthesized PEGDA capsules with 10 mM conjugated RGDS sampled at day 1.

FIG. 19C shows MSCs encapsulated in synthesized PEGDA capsules without conjugated RGDS sampled at day 7.

FIG. 19D shows MSCs encapsulated in synthesized PEGDA capsules with 10 mM conjugated RGDS sampled at day 7.

FIG. 19E shows MSCs encapsulated in synthesized PEGDA capsules without conjugated RGDS sampled at day 14.

FIG. 19F shows MSCs encapsulated in synthesized PEGDA capsules with 10 mM conjugated RGDS sampled at day 14.

FIG. 20A shows MSCs in PEGDA capsules with 10 mM RGDS and without growth factor bFGF sampled at day 1.

FIG. 20B shows MSCs in PEGDA capsules with 10 mM RGDS and with 0.25 μg/L growth factor bFGF sampled at day 1.

FIG. 20C shows MSCs in PEGDA capsules with 10 mM RGDS and with 25 μg/L growth factor bFGF sampled at day 1.

FIG. 20D shows MSCs in PEGDA capsules with 10 mM RGDS and without growth factor bFGF sampled at day 7.

FIG. 20E shows MSCs in PEGDA capsules with 10 mM RGDS and with 0.25 μg/L growth factor bFGF sampled at day 7.

FIG. 20F shows MSCs in PEGDA capsules with 10 mM RGDS and with 25 μg/L growth factor bFGF sampled at day 7.

FIG. 20G shows MSCs in PEGDA capsules with 10 mM RGDS and without growth factor bFGF sampled at day 14.

FIG. 20H shows MSCs in PEGDA capsules with 10 mM RGDS and with 0.25 μg/L growth factor bFGF sampled at day 14.

FIG. 20I shows MSCs in PEGDA capsules with 10 mM RGDS and with 25 μg/L growth factor bFGF sampled at day 14.

FIG. 21A shows MSC in nondegradable PEGDA capsules with 10 mM RGDS with 25 μg/L growth factor bFGF sampled at day 1.

FIG. 21B shows MSC in degradable PEGPQ capsules with 10 mM RGDS with 25 μg/L growth factor bFGF sampled at day 1.

FIG. 21C shows MSC in nondegradable PEGDA capsules with 10 mM RGDS with 25 μg/L growth factor bFGF sampled at day 7.

FIG. 21D shows MSC in degradable PEGPQ capsules with 10 mM RGDS with 25 μg/L growth factor bFGF sampled at day 7.

FIG. 21E shows MSC in nondegradable PEGDA capsules with 10 mM RGDS with 25 μg/L growth factor bFGF sampled at day 14.

FIG. 21F shows MSC in degradable PEGPQ capsules with 10 mM RGDS with 25 μg/L growth factor bFGF sampled at day 14.

FIG. 22A shows AtT-20 cells in PEGDA capsules without RGDS sampled at day 1.

FIG. 22B shows 3T3 cells in PEGDA capsules without RGDS sampled at day 1.

FIG. 22C shows JURKAT cells in PEGDA capsules without RGDS sampled at day 1.

FIG. 22D shows 3T3 cells in PEGDA capsules without RGDS sampled at day 7.

FIG. 22E shows JURKAT cells in PEGDA capsules without RGDS sampled at day 7.

FIG. 22F shows 3T3 cells in PEGDA capsules without RGDS sampled at day 14.

FIG. 22G shows JURKAT cells in PEGDA capsules without RGDS sampled at day 14.

FIG. 23A is a graph of cell count vs. Carboxyfluorescein succinimidyl ester (CFSE) signal of PBMCs stimulated with Soluble PHA/IL-2 in a ratio of 10 μg/mL:100 ng/mL and encapsulated in degradable PEG capsules. Cells were harvested from capsules on day 4 post stimulation.

FIG. 23B is a graph of cell count vs. CFSE signal of PBMCs stimulated with Soluble CD3/CD28 in a ratio of 50 ng/mL:50 ng/mL and encapsulated in degradable PEG capsules. Cells were harvested from capsules on day 4 post stimulation.

FIG. 23C is a graph of cell count vs. CFSE signal of PBMCs stimulated with PEGylated CD3/CD28 50 ng/mL:50 ng/mL and encapsulated in degradable PEG capsules. Cells were harvested from capsules on day 4 post stimulation.

FIG. 24 is a schematic of a cell counting apparatus and method.

FIG. 25 is a schematic illustrating a method of producing capsules.

FIG. 26 is a schematic illustrating a method of conjugating Lentivirus to PEG for in situ cell engineering.

FIG. 27 is a schematic illustrating a method of rendering PEG degradable.

FIG. 28 is a schematic illustrating a method of crosslinking and encapsulating in a molecular view.

FIG. 29 is a schematic illustrating a method of crosslinking and encapsulating in a bulk view.

FIG. 30 shows Lentivirus (LV) particles conjugated and encapsulated with HEK293T cells. RFP (Red Fluorescent Protein) in this image corresponds to a fluorophore that fluoresces red when excited, such as when a gene is in the cell of interest, confirming the occurrence of lentiviral transduction of HEK293T cells with the gene of interest and integration of the gene of interest into host HEK293 cell genome. The GFP (Green fluorescent protein) in the image corresponds to live cells, the GFP from Calcein AM staining, which is a dye used to determine cell viability because it readily permeates intact, live cells.

### DETAILED DESCRIPTION

A description of example embodiments follows.

Methods and systems of the present invention provide for encapsulated cell culture systems that can be used for expansion and/or storage of adherent cells. Such encapsulated cell culture systems and methods provide several advantages over prior art microcarriers.

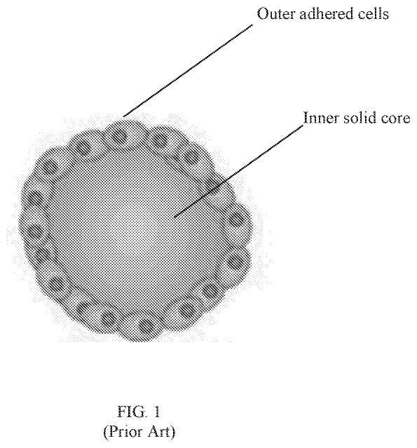

As shown in FIG. 1, prior art microcarriers provide for cell attachment on the outside of the carriers, which exposes adhered cells to fluid shear stresses in suspension cultures. Such exposure can lead to cell detachment from the microcarrier and cell damage. While cell quality is important for many bioprocessing applications, it is of particular importance for applications involving the growth and harvesting of cells for cell therapies. For such applications, the cells are the therapeutic/medicinal agent to be collected upon completion of the bioprocess, as opposed to, for example, applications in which the cells are a source for protein production and are discarded after the bioprocess.

Prior art microcarriers, while providing several advantages over 2D cell cultures for large scale bioprocessing applications, have several disadvantages, and there exists a number of challenges for their use, as summarized in Table 1 with regard to, for example, mesenchymal stem cells (MSCs). MSCs are an example of an adherent cell type, the bioprocessing of which could benefit from methods and systems of the present invention. However, it should be understood that the same or similar comparisons may be drawn with regard to other cell types, and methods and systems of the present invention may provide for or include the culturing and/or storage of a variety of different cell types, as described further. Examples of other cell types that could be input into capsules with adherent populations include Chinese Hamster Ovary (CHO) cells, Madin-Darby Canine Kidney Epithelial (MDCK) cells, HeLa cells, PC9 cells, Vero cells, pancreatic islets, peripheral blood mononuclear cells, endothelial progenitor cells, blood fibrocytes, bone marrow, T cells, B cells, dendritic cells, NK cells, monocytes, CD34+ cells, iPSC cells, EPCs, hepatocytes, neural stem cells, gastrointestinal cells, skin cells and progenitors, cancer cells, hybridoma cells, microorganisms, HEK293T packaging cell lines, yeast cells, pancreatic precursor cells, embryonic stem cells, or an induced pluripotent stem cells.

TABLE 1
Current challenges associated with the integration of microcarriers into MSC
manufacturing.

| | Benefit | Challenge | Potential Mitigation |
|---|---|---|---|
| Product | Reduced labor requirements and | Quality: High risk of | Development of quality |
| Comparability | ability to implement automated | changes in MSC | assays of product |
| | process steps. | product efficacy when | efficacy. |
| | Stirred bioreactors are well | moving from existing | |
| | characterized, allowing for changes | planar technology to | |
| | in scale to be assessed prior to | microcarrier-based | |
| | implementation [21]. | process. | |
| | Reduction in required clean room | | |
| | capacity and equipment (incubators | | |
| | etc.) allowing for facility and | | |
| | operational cost savings. | | |
| Product Yield | Ability to manufacture >10⁹ cells to | Cost of Goods: | Development and |
| (per | provide >4000 doses per batch compared | Increased MSC | optimization of culture |
| bioreactor | with <10⁸ cells to provide <400 doses per | number per batch | medium and process |
| volume) | batch in planar culture technology [22]. | reduces the cost per | parameters |
| | | dose. | |
| Attachment | Required for anchorage-dependent cells | Sustainability: | Development of reduced |
| | to maintian cell phenotype and | Reducing the amount | or serum-free process |
| | pluripotency in the case of stem cells. | of serum is critical in | including methods for |
| | | increasing large-scale | microcarrier modification |
| | | sustainability. | to improve MSC |
| | | Cost of Goods: | attachment [23]. |
| | | Reduced attachment | |
| | | reduces growth rate | |
| | | and number of MSCs | |
| | | per batch. | |
| Harvest | Reduction in batch pooling and product | Quality: Increased | Development of MSC |
| | holding times, improving process | agitation rate during | detachment protocols |
| | scalability and product quality attributes | detachment reduces | prior to implementation |
| | [24]. | product quality. | at scale [25, 26]. |
| | | Cost of Goods: | |
| | | Increased detachment | |
| | | efficiency and MSCs | |
| | | per batch reduces the | |
| | | cost per dose. | |
| Separation | Improved integration of up- and down- | Quality: Method of | Development of scale- |
| | stream unit operations, reducing process | separation may affect | down models to test |
| | time and improving scalability. | MSC CQAs | product CQAs [27] and |
| | | Scalability: Process | downstream processing |
| | | should allow for timely | times early in |
| | | separation, even at | development [23]. |
| | | large scale. | |
| Process | Homogeneous environment allows | Quality: Impact on | Integration of effective |
| Optimization | for monitoring and control and key | MSC product CQAs. | process control systems, |
| | process parameters such as | Scalability: Limit to | media optimization and |
| | dissolved oxygen, pH, nutrients and | process scale. | consideration of direct |
| | metabolites[28]. | | aeration methods [13] in |
| | Bioreactor systems allow for flexible | | early development. |
| | modes of operation such as batch, | | |
| | fed-batch or perfusion, allowing for | | |
| | process development activities to | | |
| | improve product quality and yield. | | |
| Purification | Closed system manufacture, reducing the | Quality: Increase in | Integration of particulate |
| | risk associated with contamination and | rate of failed lots due | and impurity levels as a |
| | failed product lots. | to impurities. | screening criterion for |
| | | | development of |
| | | | downstream separation |
| | | | and volume reduction |
| | | | processes. |

In suspension cultures, fluid shear advantageously prevents carrier sedimentation and cell aggregation while ensuring cell culture homogeneity; however, shear stress affects cell viability and morphology and can have a modulating effect on cell metabolism and differentiation states [29]. Moreover, shear stress from agitation results in cell damage due to microcarrier-to-microcarrier or microcarrier-to-impeller (or probe/insert) collisions. This damage increases with increasing microcarrier size, concentration, and agitation intensity, incurring a limitation on scale-up [30]. Smaller microcarriers can reduce shear-induced cell death and increase growth rates; however, decreasing the size of the microcarriers likewise decreases the available surface area for cell attachment and expansion. A minimum agitation rate has been estimated from an empirical correlation derived by Zwietering [101], which suggests that microcarriers should not remain at the vessel bottom for more than 1-2 s. Taking in view the Kolmogorov model [102], turbulent eddies in stirred-tank bioreactor microcarrier cultures are intermediate in size between the cells and the microcarriers. The Kolmogorov eddy size decreases as the agitation speed increases. The high rate of local energy dissipation due to these eddies interacting with the surface of microcarriers can cause shear rates that are sufficiently large to damage or even remove cells from the microcarrier surface. Additionally, foam formation from aeration leads to hydrodynamic stress in large-scale cultures, which is detrimental to cells growing on the surface of microcarriers [13, 31]. At the cellular level, exposure to a rigid microcarrier bead and shear fluid flow patterns can have dramatic impact on cell viability and mechanotransduction ultimately impacting the quality of cultured cells.

Efforts are beginning to direct towards protection of cells from shear forces expanded using the microcarrier technology; porous carriers such as the Cultisphere® (Sigma Aldrich, UK) have been developed that shield cells from shear-induced damage and allow higher cell growth through colonization of cells in the pores of the carrier [32-34]. Though a plausible solution, another important issue, identified by Gupta et al. [35], is that agitation of Cytodex® 3 microcarrier cultures (GE Healthcare, Marlborough, Mass.) above 25 rpm results in carrier breakage. As such, use of soft carriers, such as Cytodex® 1/3 (GE Healthcare, Marlborough, Mass.), raises safety concerns for in vivo applications where cells need to be free of any residual microcarriers or sub-particles. Current approaches to cell harvesting involve the use of cell strainers to separate expanded cells from their carriers; however, the breakage products are difficult to remove. These carrier impurities continue to be of concern and are a major obstacle for the clinical application of cell therapeutics cultured on microcarriers.

Methods and systems of the present invention provide alternatives to currently-available microcarriers that can circumvent one or more of the above-noted deficiencies, such as, for example, by promoting increased viability and purity of the final cell product.

In one embodiment, the invention is directed to a capsule for growing or storing cells that includes a shell defining an interior compartment and a substrate for cell attachment located within the interior compartment. The substrate comprises a polymer and one or more adhesion molecules. The adhesion molecules, also referred to as adhesion conjugates, can be, for example, adhesion peptides, adhesion proteins, or small molecules capable of attaching cells to a substrate and/or activating a differentiation pattern of attachment. For example, adhesive conjugates can be proteins (e.g., partial or full-length proteins), such as collagen type I, fibronectin, laminin, collagen type IV, Matrigel, or any combinations thereof. Adhesive conjugates can also be antibodies, which engage with cell surface receptors, such as T cell receptors. For example, the antibody can be an antibody that binds at least one of CD3, CD28, and CD40. Adhesive conjugates can also be or include nonpeptide small molecules that activate a differentiation program in cells to enhance adhesion, such as stemregulin or reversine, or a non-steroidal anti-inflammatory molecule.

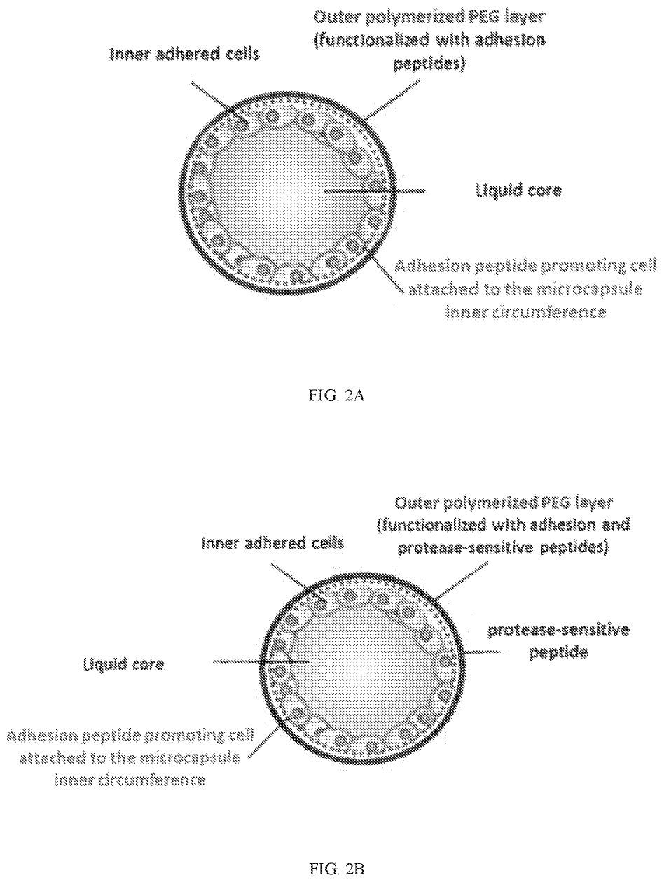

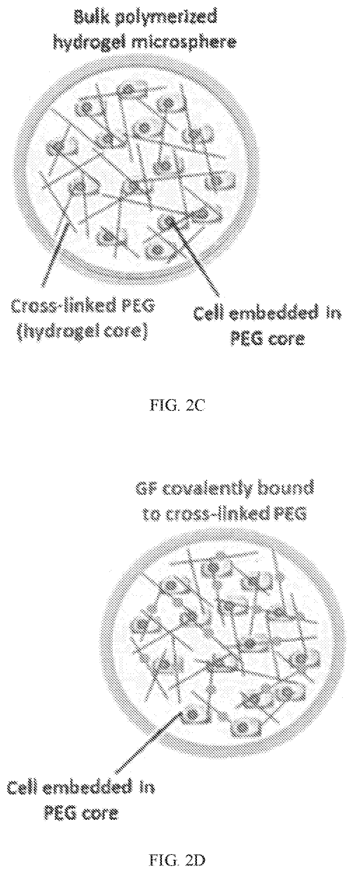

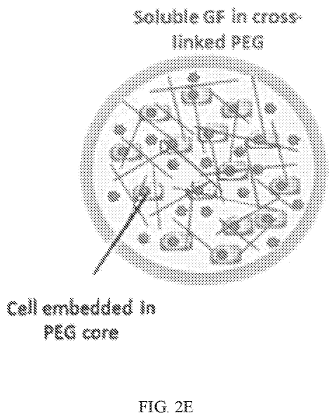

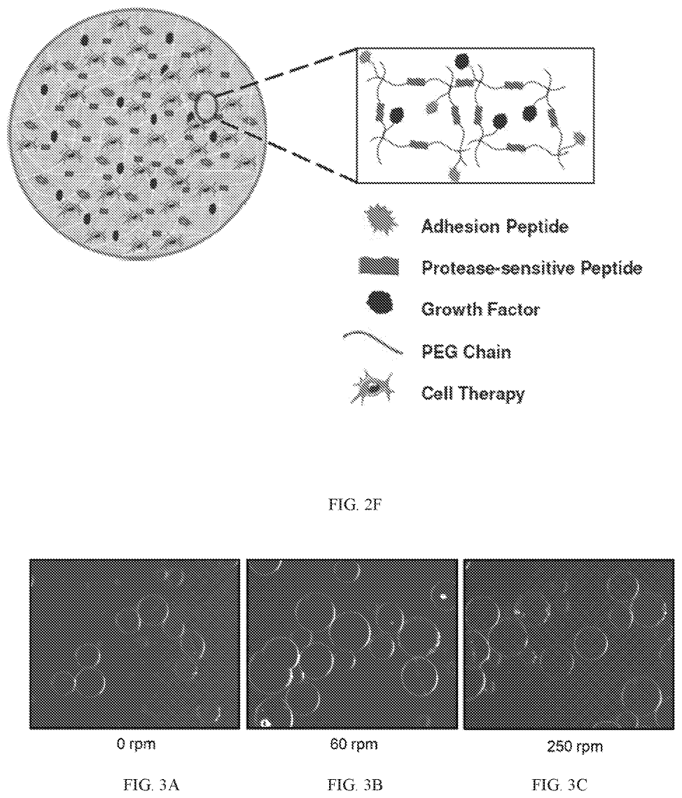

Examples of capsules are shown in FIGS. 2A-2E. The substrate for cell attachment can be, for example, an inner surface of the shell, as shown in FIGS. 2A-2B, such that cells are attached to the capsule along an inner circumference of the capsule. Alternatively, or in addition, the substrate for cell attachment can be a hydrogel disposed within the interior compartment of the capsule, as shown in FIGS. 2C-2E.

The encapsulation of cells, such as MSCs, protects the cells from mechanical agitation and shear stresses while an interior of the capsule provides for a surface area onto which the cells may adhere and a space into which the cells may expand as cell clusters. In some embodiments, the shell comprises a natural or synthetic polymer. The polymer can be, for example, polyethylene glycol (PEG), polyethylene glycol diacrylate (PEGDA), alginate, chitosan, PEG copolymerized with alginate or chitosan, and PEGDA copolymerized with alginate or chitosan, or any combination thereof. Other polymers include PLGA, PLL, PDMS, polyacrylamide, polyacrylamide, poly(N-isopropylacrylamide) (PNIPAAm), poly[2-(methyacryloxy)ethyl phosphorylcholine]-block-(glycerol monomethacrylate) (PMPC-PGMA), x-acetylene-poly(tert-butyl acrylate) (PtBA), poly(vinyl alcohol) (PVA), poly(acrylic acid) (PAA), poly(divinylbenzene-co-glycidylamethacrylate) (P(DVB-GMA)), poly(amidoamine) (PAMAM), poly(D-glucosamidoethylenemethacrylate) (PGAMA), poly(2-lactobionamido ethylmethacrylate (PLAMA), alkyl thioether end-functionalized poly(methacrylic acid) (PMAA-DDT), poly(ethylene glycol)-phosphine (PEG-phosphine), poly(vinyl pyrollidone) (PVP), poly(acrylic acid)-octylamine (PAA-octylamine), poly(maleic anhydride-alt-1-octadecene-block-poly(ethylene glycol) (PMAO-PEG).

With regard to materials for cell encapsulation, several natural and synthetic polymers have been investigated in rodent models; with alginate and polyethylene glycol (PEG) reaching pre-clinical and clinical trials. Alginate provides favorable gelling conditions; however, challenges associated with batch to batch variability, wide pore size distribution, encapsulated product size (e.g., engraftment volume) and scalability exist [10, 11]. PEG has been used for conformal coats on islets because it can react with amine groups in collagen and membrane proteins on the islet surface [23-25], which has the advantage of shorter distance for diffusion for oxygen, nutrients, and insulin and a smaller implantation site. Moreover, PEG can be functionalized with peptides and growth factors to stimulate insulin gene transcription, prevent (3-cells apoptosis and promote islet vascularization [31-33].

A comparison of methods and lead materials for producing encapsulated cells is shown in Table 2. While alginate and PEG are provided as examples, other materials that can be polymerized could potentially be combined with the encapsulation methods described herein, or other known encapsulation methods, for polymer matrix components. For example, one method of producing small monodisperse capsules in a high-throughput manner includes the pulsation of a jet or vibration of a nozzle during extrusion of the lead material, often referred to as a laminar jet breakup technique. The laminar jet breakup technique involves axisymmetric disturbances to break the jet from the nozzle into equally sized droplets. This technique can achieve production rates as high as 104 particles per second [22]. The vibration frequency, diameter of the nozzle, viscosity, and flow rate of the polymer-cell suspension govern the size and production rate of the microcapsules. In another example, a rotating disk or jet cutting method can be used to produce small monodisperse capsules. Jet cutting can provide a higher production rate for generating particles with the encapsulated material at a frequency of 10,000 Hz, or 104 particles per second, which translates to a 500 μm bead throughput of 60 mL/min [22].

Other methods include emulsification techniques, which, compared to extrusion drip methods, are not limited by scale. With emulsification techniques, a production rate is governed by the vessel size in which cell encapsulation takes place. Emulsification techniques also provide for the ability to produce cell-laden microcarriers in a single step as compared to the two-step procedure required with commercially available microcarriers. Appropriate dispersion devices and operating conditions, such as mixing rates and surfactants, can allow for reduction in capsule size.

TABLE 2
Integration of lead materials candidates with high throughput methods of
producing encapsulated cells.

| Encapsulation | | | | | |
|---|---|---|---|---|---|
| Technique | Scalability | Production Rate | Material | Characterization | Ref |
| Extrusion | Medium to | Coaxial air flow: 10² part./s | Alginate | In vitro, rodent, | [40- |
| | high | Electrostatics: 10⁵ part./s | | dog, nonhuman | 52] |
| | | Vibration: 10³ part./s | | primate, human | |
| | | JetCutter: 10⁴ part./s | | | |
| Emulsion | High | Depends on vessel size | PEG/PEGDA | In vitro, rodent, | [53- |
| | | | | nonhuman | 62] |
| | | | | primate, human | [63- |
| | | | | | 65] |
| | | | Alginate | In vitro, rodent | |
| | | | Agarose | In vitro, rodent, | |
| | | | | dog | |
| Microfluidic | Medium | 10⁴ particles/s | Alginate | In vitro, rodent | [66, |
| | | | PEG | | 67] |
| Surface coat | Low | Depends on coating | PEG/PEGDA | In vitro, rodent, | [68- |
| | | method | | human | 70] |

In further embodiments, in addition to a polymer shell, capsules can further include a hydrogel core, as shown, for example, in FIGS. 2D-2F. The hydrogel core can comprise, for example, cross-linked polyethylene glycol (PEG), cross-linked polyethylene glycol diacrylate (PEGDA), or a combination thereof.

The polymer shell and/or the hydrogel core can be functionalized with one or more adhesion peptides that allow for cell attachment and/or spreading within the capsule. The adhesion peptides can be, for example, RGDS (SEQ ID NO:1), YIGSR (SEQ ID NO:2), IKVAV (SEQ ID NO:3), REDV (SEQ ID NO:4), GKKQRFRHRNRKG (SEQ ID NO:5), RNIAEIIKDI (SEQ ID NO:6), KTRWYSMKKTTMKIIPFNR (SEQ ID NO:7), or any combination thereof. Adhesion peptides can be peptides that affect cell viability, proliferation, survival, growth and/or differentiation.

For example, PEG can be chemically modified to include one or more adhesion peptides by reacting acrylate-PEG-SVA with an adhesion peptide in a sodium bicarbonate solution at a predefined molar ratio (e.g., 50 mM, pH8.5) overnight, followed by dialyzation to remove any unreacted peptides. The modified polymer material can, optionally, be lyophilized and stored. Adhesion peptide conjugation efficiency can be assessed by detecting any unreacted free amines by a ninhydrin assay. Acceptable conjugation efficiency can be determined based upon the desired application. For example, a conjugation efficiency of about 85% or greater could be considered acceptable. Examples of adhesion peptides, protein derivatives, and resulting PEGylated products are shown in Table 3.

Table 3. Candidate adhesion peptides for PEGylation

| | | | Molar Ratio | |
|---|---|---|---|---|
| PEGylation | Adhesion | Protein | (PEG: | PEGylated |
| ester | Peptides | Derivative | peptide) | Product |
| Acryl-PEG-SVA | Viability [71,72] | | | |
| 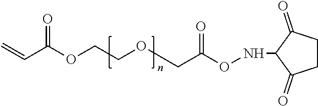 | RGDS (SEQ ID NO: 1) YIGSR (SEQ ID NO: 2) IKVAV | Fibronectin Laminin Laminin | 1:1.2 | PEG-RGDS PEG-YIGSR |
| | (SEQ ID | | | PEG-IKVAV |
| | NO: 3) | | | |
| | Undifferentiated growth [73] | | | |
| | GKKQRFR | Vitronectin | 1:4 | PEG-QRFR |
| | HRNRKG | (heparin | | |
| | (SEQ ID | binding: | | |
| | NO: 5) | promote GF | | |
| | | signaling) | | |
| | Proliferation [74] | | | |
| | RNIAEIIKDI | Laminin | 1:1.2 | PEG-AEII |
| | (SEQ ID | | | |
| | NO: 6) | | | |
| | No effect on cell survival or proliferation rate [75] | | | |
| | KTRWYSM | Fibrinogen | 1:5 | PEG-RWYS |
| | KKTTMKIIP | | | |
| | FNR (SEQ | | | |
| | ID NO: 7) | | | |

Conjugation Efficiency

| | |
|---|---|
| Ninhydrin Assay | Access peptide conjugation effiency to |
| | PEG (>85% accepted) |

Encapsulation Efficiency

| | |
|---|---|
| MTS Assay | Cell encapsulation efficiency |
| | (>60% accepted) |

Attachment Efficiency

| | |
|---|---|
| Flourescent Microscopy | Assess actin organization |

The conjugated adhesion peptides (e.g., PEG-conjugated adhesion peptides, or acryl-PEG-AP) can be combined with a cell suspension (e.g., an MSC suspension) prior to undergoing a photopolymerization process (e.g., by being added to photopolymerizable PEG diacrylate (PEGDA) and undergoing free-radical polymerization) to form cell-laden PEG capsules. Concentrations and combinations of acryl-PEG-AP can be varied in the precursor solution to optimize cell attachment. For example, hydrogels can be formed by combining 0.1 g/mL 10 kDa PEGDA and 10±5 mM acryl-PEG-AP in 10 mM HEPES buffered saline (pH 7.4) and photoinitiators 1.5% v/v triethanolamine (TEOA), 37 mM 1-Vinyl-2-pyrrolidinone (NVP), and 10 μM eosin Y disodium salt. The solution can then be sterilized by being filtered, such as through a 0.2 μm filter. By combining the cells (e.g., MSCs) in the precursor solution, the cells can be encapsulated during photopolymerization and formation of the capsules. In an example procedure, a hydrophobic photoinitiator solution containing 2,2-dimethoxy-2-phenyl acetophenone in 1-vinyl-2-pyrrolidinone (300 mg/mL) can be combined in mineral oil (3 μL/mL), to which the cell-prepolymer suspension is added. The combined solution can then be vortexed for 4 seconds in ambient light, followed by an additional 3 seconds under white light. The vortex may then be stopped and the emulsion exposed to white light for 20 seconds with a vortex pulse at 10 seconds. Crosslinked microspheres can then be isolated by centrifugation at 300 g for 5 minutes, resuspended in media, and placed in Transwell® (Corning, Tewksbury, Mass.) cell culture inserts.

Morphological evaluation of the encapsulated cells and polymer shells/hydrogel core can be conducted using fluorescent stains. In an example of an evaluation procedure, images can be taken to verify the interaction of cells and the surrounding hydrogel matrix. Actin organization of the encapsulated MSCs can be analyzed to verify bioactivity. Upon encapsulation, MSCs may be attached along the inner membrane of the PEG capsules, anchoring to the PEGylated adhesion moieties. Cell-laden capsules (alternatively referred to as microcapsules) can be fixed in a 4% formaldehyde solution, permeabilized using a 1% Triton X-100 solution, then incubated for 30 min in a 4 unit/ml phalloidin rhodamine solution and mounted in a mounting solution containing a fluorescent stain, such as DAPI. Cell adhesion and spreading can be monitored by, for example, light microscopy and fluorescence microscopy, such as on an Axio Observer Z1 (Zeiss, Jena, Germany) equipped with an ApoTome system (Zeiss, Jena, Germany) to achieve optical sectioning. Selection of an adhesion peptide can be made by semi-quantitative analysis of, for example, 100 pictures of random cell capsules for N=3 batches per peptide to quantify spindle formation as compared to no peptide controls. An acceptable attachment efficiency can be determined based upon the desired application. For example, an attachment efficiency of at least about 60% could be considered acceptable. Alternatively, a desired adhesion peptide can be selected based on other factors. For example, it may be desirable to include RGDS because RGDS is well characterized for directing cell association with biomaterials [76-78].

While an example of forming encapsulated cells by photopolymerizing a precursor solution containing cells is described, other methods of encapsulating cells are possible. For example, a capsule can include a porous shell and/or a porous hydrogel core. The porous capsule can then be exposed to a cell suspension such that cells translocate into the interior compartment of the shell and adhere to the substrate.

In one embodiment, a capsule includes a porous shell having a pore size of about 10 nm to about 35 nm (e.g., 0.9 nm, 10 nm, 15 nm, 20 nm, 25 nm, 30 nm, 35 nm, 35.5 nm), or of about 20 nm.

Capsules having a pore size of about 10 nm or greater, or of about 20 nm or greater, can also allow for the diffusion of nutrients and waste products into and out of the capsules. A pore size can be adjusted by modifying a weight of the polymer precursor material used to create the hydrogel mesh that forms the shell and/or hydrogel core of the capsule. For example, PEG-SVA having a weight of 5 kDA can be used to form a hydrogel mesh that has a larger pore size than that of a hydrogel mesh made from 10 kDa PEG-SVA.

In another embodiment, capsules can include one or more growth factors. The inclusion of a growth factor can further enhance cell growth and/or proliferation within the capsule. The one or more growth factors can be conjugated to the polymer (e.g., the polymer shell, the hydrogel core, or both). Alternatively, the growth factor(s) can be soluble within a liquid core or a hydrogel core of the capsule. Examples of suitable growth factors include FGF, TGF-β1, VEGF, PDGF-BB, PDGF, IGF1, and BMP superfamily members, or any combination thereof.

Growth factors (GFs) may be required for some in vitro cell cultures and are typically added to culture media as soluble GFs. Capsules of the present invention can advantageously provide for a concentrated, transient, controlled supply of bioactive GFs and reduce the need for serum components typically used as a growth agent for cultured cells. For example, PEGylated GFs can be included in the shell and/or core of a capsule. Examples of PEGylated GFs, including FGF, TGF-β1 [79] [80], IGF1[81] and BMP [82] [83], are shown in Table 4.

TABLE 4
Table 4: Growth Factor PEGylation

| PEGylation | Growth | Molar Ratio | PEGylated |
|---|---|---|---|
| ester | Factor | (peptide: PEG) | Product |
| Acryl-PEG-SVA | FGF | 1:15 | PEG-FGF |
| 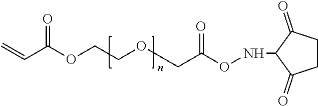 | TFG-β1 IGF1 BMP | 1:19 1:21 1:20 | PEG-TGF PEG-IGF PEG-BMP |

PEGylation Evaluation
Western Blot
GF Bioactivity Evaluation

| | |
|---|---|
| Cell Proliferation Assay | Experimental Groups |
| | (i) soluable GF, (ii) PEG-GF, (iii) control: |
| | no GF |
| | Outcome: Cell counts |

In an example procedure, recombinant GFs can be conjugated to PEG by reaction with acrylate-PEG-SVA in a 1:15 (peptide:PEG) molar ratio in 50 mM sodium bicarbonate (pH 8.5) and then stirred under argon overnight, lyophilized, and stored at −80° C. A Western blot can be used to analyze the resulting acryl-PEG-GF. Soluble and PEG-conjugated GF can be separated on a 4-15% SDS-PAGE gel and transferred to a nitrocellulose membrane. The membrane can be incubated overnight at 4° C. with 5% milk in buffer containing 0.1% (vol/vol) Tween 20 in TBS (TBST). The membrane can be incubated with rabbit anti-GF for 1 h at room temperature. After two washes with TB ST, peroxidase-labeled goat anti-rabbit IgG can be added and incubated for 1 h at room temperature and treated with pico-chemiluminescence reagent for detection. The bioactivity of the PEG-GF conjugates can be assessed to verify that bioactivity was not affected by the conjugation process or due to steric hindrance. The PEG-GF conjugates can be assessed in capsules at a range of concentrations of GF (e.g., 1 μg/L, 2.5 μg/L or 5 μg/L.) Cell growth can be measured in the presence of (i) unmodified or soluble GF, and (ii) PEG-conjugated GF to determine suitability of conjugated GF for an application. For example, a 50% growth advantage by PEG-GF vs PEG acceptable growth advantage by PEG-GF versus PEG may be desirable. The MSCs can be seeded in 12-well plates at an estimated density of 5000 cells/capsule and incubated for a 7 day growth promotion assay in a 37° C./5% CO2 environment. After 7 days, cell capsules can be quantified by imaging and MTS assays to assess cell growth. Additional supplementation of GF to a cell culture media may not be needed in capsules comprising PEG-GF.

As noted above, the purification of cells from prior art microcarriers can be challenging, often resulting in product having residual microcarriers and subparticles present. To address this shortcoming, PEG hydrogels can include proteolytically degradable peptide sequences to control hydrogel biodegradation. In one embodiment, capsules of the present invention include an enzyme-sensitive, such as a protease-sensitive peptide (e.g., GGGPQG↓IWGQGK (SEQ ID NO:8), GGL↓GPAGGK, GGG↓LGPAGGK (SEQ ID NO:9). The enzyme-sensitive peptide can be included in the shell and/or in a hydrogel core of the capsule. After a cell expansion process has completed, the capsule material can be caused to degrade, and standard cell concentration methods can then be used to purify the cell material. Some examples of enzymes used are trypsin, collagenase, DNase, RNase, and horseradish peroxidase. Other sensitive structures can be doped into capsules that infer temperature sensitivity, light sensitivity, or small molecule sensitivity, as well as protein-based sensitivity. For example, covalent bulk immobilization of PEGylated GF(s) in a degradable PEG hydrogel construct can provide a controlled transient local release of GF(s) to stimulate MSC growth and expansion while also providing for capsule degradation. Examples of degradable PEG are shown in Table 5.

TABLE 5
Table 5: Degradable PEG
Enzymatic degradation

| | | Molar | |
|---|---|---|---|
| | | Ratio | |
| PEGylation | Degradable | (PEG: | PEGylated |
| ester | Peptide | peptide) | Product |
| Acryl-PEG-SVA | GGGPQG↓I | 2:1 | Single domain: |
| 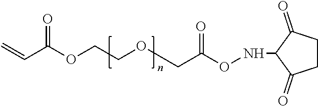 | WGQGK (SEQ ID NO: 8) | | acryl-PEG- peptide- PEG-acryl Multi domain: acryl-(PEG- peptide)₃- PEG-acryl |

Non-enzymatic degradation
Tyrosine derived PEG carbonates/ethers
Glutathione-degradable PEG-thiol
Degradation Process Controls
Conc. of degradation agent, Time, Temperature, Neutralization (>95% dissolution by
weight accepted)

Hydrogels can be rendered degradable through, for example, the covalent incorporation of a collagenase-sensitive peptide sequence, such as GGGPQG↓IWGQGK (SEQ ID NO:8), (PQ), where ↓ indicates a cleavage point by collagenase between the leucine and glycine residues. Single and multisite PQ domains can be incorporated between acrylate groups. Example procedures for forming degradable capsules follow. For single domain, the PQ peptide can be dissolved in 50 mM NaHCO₃ (pH 8.0) and reacted with PEG-SVA (MW=3400 Da) in a 1:2 (PQ: PEG-SVA) molar ratio at room temperature overnight to attach PEG on both ends of the peptide sequence (PEG-PQ-PEG). The resulting products, acrylate-PEG-peptide-PEG-acrylate can be dialyzed, lyophilized, and stored frozen at −20° C. under argon. For multidomain PQ conjugation, the peptide can be reacted with acryl-PEG-SVA in equal molar ratios, then dialyzed and lyophilized to remove undesired products. This acryl-PEG-peptide can be further reacted with SVA-PEG-SVA (MW 3400 Da), dialyzed and lyophilized. The reaction step can be repeated to add additional PEG-peptide. In the final step, the previous product can be reacted with acryl-PEG-SVA to form the multisite PQ degradable PEGDA macromer. The final product acrylate-(PEG-peptide)3-PEG-acrylate can be dialyzed, lyophilized and stored at −20° C.

The rate of MSC invasion within collagenase-sensitive PEG hydrogels can be evaluated to determine the conduciveness of the microcapsule to MSC expansion within the degradable substrate with local access to GF(s). Hydrogel degradation kinetics can be determined by, for example, allowing the microcapsules to swell for 24 hrs in PBS with 1 mM CaCl₂ at 37° C. Microcapsules can then be incubated at 37, 33 or 25° C. with collagenase from Clostridium hystolyticum in PBS with 1 mM CaCl₂. The change in wet weight of the microcapsules can be measured over time. Degradation conditions in terms of enzyme concentration, time, temperature, and neutralization can be determined.

Non-enzymatic methods of polymer degradation can also be included in methods and systems of the present invention. For example, PEG segments can be incorporated into tyrosine-derived polycarbonates, as described by Kohn et al. [84-86], and/or thiol functionalized PEG macromers can be used to provide degradability in response to reducing microenvironments, such as in the presence of glutathione [87]. This degradation can occur via a thiol-disulfide exchange reaction. This reaction will fragment the polymer into soluble units [103]. As such the microcapsules will be prepared by employing 4-arm PEG or PEG tetra acrylate (PEGTA) in which each arm will be terminated with a thiol group [104] to form PEG-diester-dithiol cross-linker. The presence of disulphide bonds will allow hydrogel degradation in the presence of glutathione. Alternatively, water soluble PEG microcapsules will also be prepared by reacting PEGTA with dithiotriethol (DTT) in 1:1 molar ratio of acrylate to thiol in triethanolamine (TEA) and allowed to gel at 37° C. [105, 106]. Degradation of the capsules will occur overtime as water breaks down the hydrolytically labile ester bonds where the degradation kinetics will be dependent on reaction [105]. To attach peptides such as cell-adhesive ligands, 4-arm PEG-vinyl sulfone (PEG-VS) will be dissolved in TEA buffer and the cysteine-terminating peptides added at a large stoichiometric deficit to VS groups to covalently attach to VS [106] [107].

In addition to soluble factors, such as growth factors, and biochemical cues, including adhesion peptides, cells may also be impacted by physical and mechanical cues, such as a surface topography and rigidity/stiffness of the substrate. Capsules can be further optimized to have a modulus that retains multipotency during cell expansion.

The rationale for optimizing the spatial organization of cell adhesion peptides and mechanical stiffness of the substrate is that such characteristics can play significant roles in regulating multipotent cell function [88-90]. With regard to microcarriers, characteristics such as stiffness and curvature influence cellular activities while the adhered cells are being cultured in a bioreactor through changes in the shear stress on cells in a bioreactor [91]. Thus far, limited efforts have been made to define the role of microcarrier properties and geometry on controlling multipotent cell function [92, 93]. The regulation of MSC cell fate is dependent on substrate rigidity, which modulates the extracellular matrix (ECM) of the MSC, adhesion peptides (e.g., integrin), and cytoskeleton interactions. A failure to control for these properties can lead to unwanted differentiation of MSCs and a loss of immunomodulatory potency.

The modulus of the substrate of capsules of the present invention can be tunable. For example, polymer molecular weights and concentrations in precursor solutions can be adjusted to provide for softer/harder substrates. In one example, 3400 Da PEG-SVA and 10% 10 kDa PEGDA can be used for microcapsule formation. Cell attachment and spreading can be analyzed and the polymer weight can be decreased if the matrix is too ‘soft’ for cell attachment. Several molecular weight PEGDA (e.g., 3.4 kDa and 5 kDa) can be utilized, and the concentration can be varied (e.g., 10% or 15%) to tailor substrate modulus to cell culture and reactor conditions in future aims. To determine if the stiffness of a substrate is suitable, cell proliferation with microcapsules can be followed for 14 days and measured via MTS assay at predetermined time points. Alternatively, a desired weight/concentration of polymer precursor materials can be selected based on other factors. For example, it may be desirable to include 10% 10 kDa PEGDA because such a combination provides a balance between substrate rigidity, porosity and degradation kinetics.

In some embodiments capsules of the present invention include a cell adhered to the substrate, such as, for example, a stem cell. While the Mesenchymal Stem Cell (MSC) has been used as an example of an adherent cell that can be encapsulated within capsules of the present invention, it should be understood that other adherent cells can alternatively be included. In addition to an MSC, examples of suitable cells include Chinese Hamster Ovary (CHO) cells, Madin-Darby Canine Kidney Epithelial (MDCK) cells, Vero cells, pancreatic islet, pancreatic precursor cells, embryonic stem cells, and pluripotent stem cells. Cells contained in capsules can be maintained in a viable state, such as by being maintained in a suspension culture. Encapsulated cells can also be stored for later use, such as by undergoing a cryopreservation processes.

Cell Counting Method & Apparatus

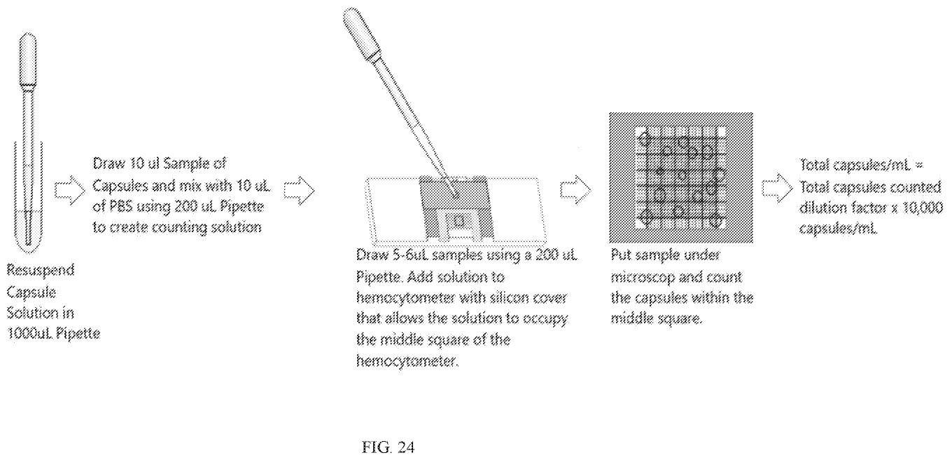

An example of a cell counting method and apparatus is shown in FIG. 24. An amount of solution containing suspended capsules can be drawn with a pipette and mixed with phosphate buffered saline (PBS) to create a counting solution. For example, 10 μL of sample can be drawn and mixed with 10 μL of PBS using a 200 μL pipette, as shown. The drawn samples can be placed in a hemocytometer with a silicon cover. For example, 5-6 μL samples can be drawn and placed in the hemocytometer with the solution occupying the middle square of the hemocytometer. Either four outer squares can be used for counting, or the middle square can be used. The middle square is shown as being used in the figure to confine capsules in area of known dimensions.

The sample can then be placed under a microscope, and capsules within the middle square counted. A total number of capsules/mL is equal to the number of capsules counted multiplied by the dilution factor and multiplied by 10,000 capsules/mL. Use of a slip cover may cause capsules to break. Silicone can be used to keep the solution in place instead of use of a slip cover.

Continuous Flow Capsule Production Using Light-Based Polymerization

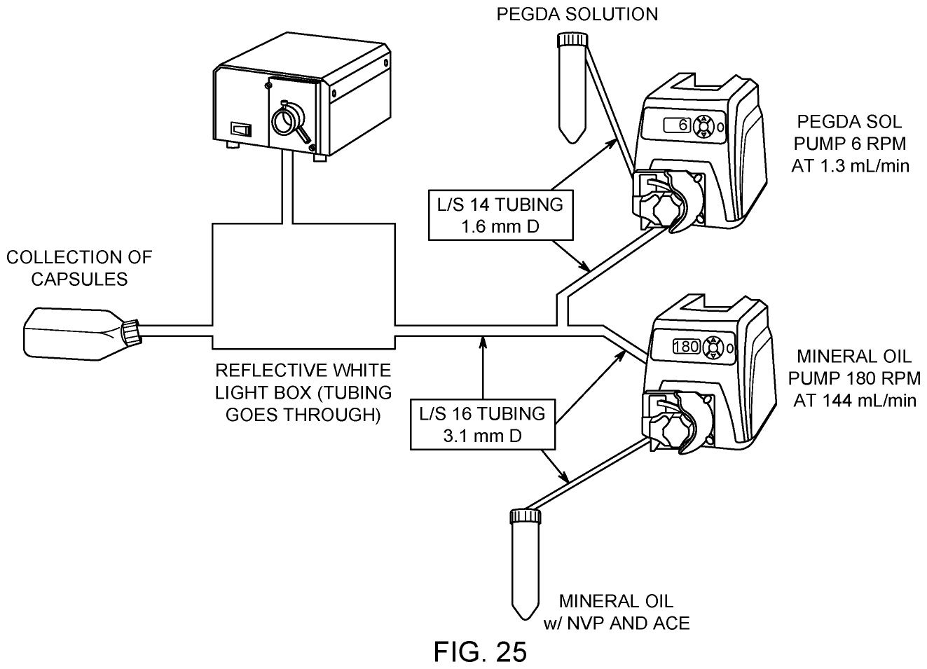

An example of a continuous capsule production system is shown in FIG. 25. A polymer solution containing a hydrophilic photoinitiator is mixed with a hydrophobic photoinitiator in mineral oil. The terms hydrophilic and hydrophobic are used to designate the solution in which the photo initiators are present. The mineral oil solution (e.g., as illustrated, 54, acetophenone/NVP solution and 1 mL mineral oil) is hydrophobic. The PEGDA solution (e.g., as illustrated, 0.1 g PEGDA, 15 uL TEOA, 10 uL Eosin Y disodium salt, 3.754, NVP, 493.025 uL HBS Solution, and 10 uL Pluronic acid) is hydrophilic.

The polymer solution can contain the polymer (e.g., PEG), cells (e.g., NK cells, MSCs, CHOs), adhesive peptides (e.g., RGDS), and, optionally, other factors, such as growth factors (e.g., FGF), viral particles, and degradation peptides (e.g., PQ peptide).

In the system illustrated in FIG. 25, a PEGDA pump pumps PEGDA solution to a reflective white light box while a mineral oil pump pumps mineral oil with hydrophobic photoinitiator to the white light box. The pumps and the reflective white light box can be connected by tubing. The white light box is connected to an illuminator.

The tubing passes through the white light box to a receptacle for storing the processed capsules. For example, the capsules can be collected in a 75 cm² T flask as the system processes capsules on a continuous basis continuous system.

The amount of time the solutions are present in the white light box can be dependent upon a number of light sources used with the reflective box, type of light sources, and setup of the reflective box. A pump speed can be adjusted accordingly to ensure that the solutions are exposed to light for an appropriate amount of time. For example, with system shown in FIG. 25, having one light source, the solution can be present in the white light box for approximately three minutes. Other configurations are possible, examples of which follow:
Mineral Oil 144 mL/min and PEDGA 1.3 mL/min with 3 minutes of light exposure.
Mineral Oil 144 mL/min and PEDGA 1.3 mL/min without using the light box and point blank exposing the mixed solution tube for 15 seconds for each inch of tubing.
Mineral Oil 96 mL/min and and PEDGA 1.3 mL/min without using the light box and point blank exposing the mixed solution tube for 15 seconds for each inch of the tubing.

Pump speed can also affect capsule size. Increasing the pump speed of the mineral oil solution can reduce the size of the PEDGA solution when both the solutions are mixed. This can allow for a smaller capsule size.

Conjugation of Lentivirus to PEG for In Situ Cell Engineering

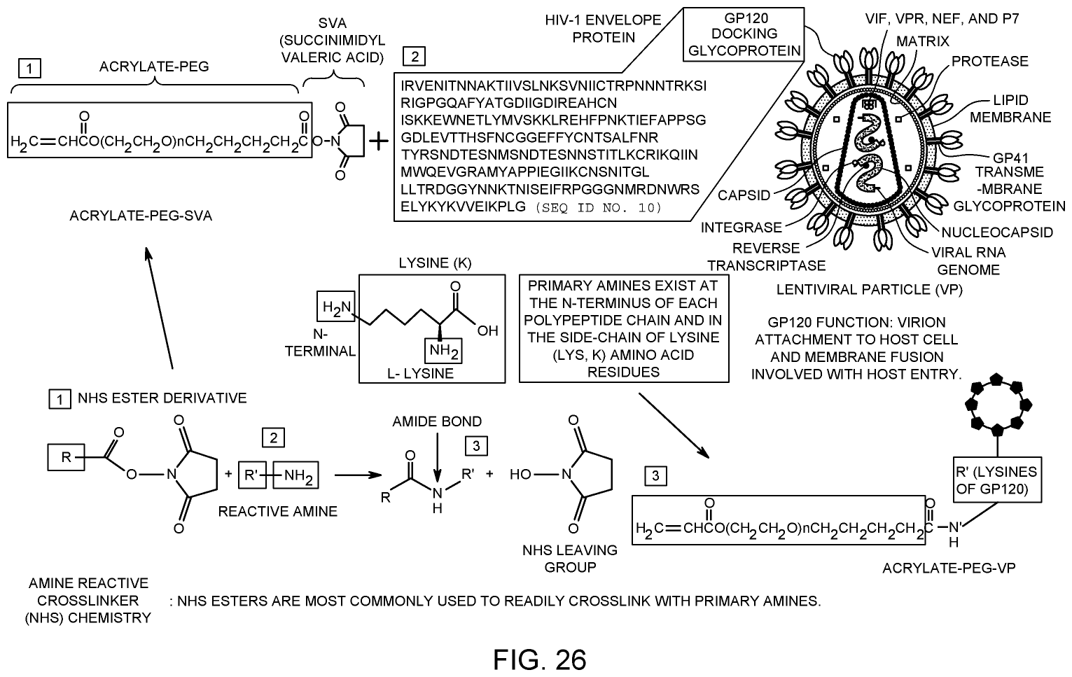

An example of a method for creating capsules comprising conjugated lentivirus is shown in FIGS. 26-29. As shown in FIG. 26, a first part of the process includes conjugation of lentiviral particles to PEG. The conjugation can be accomplished with, for example, amine crosslinker (NETS) reactive chemistry by which Acrylate-PEG-SVA (molecular weight (MW): 3,400) reacts with surface-exposed primary amines on lysine residues, part of the viral capsid glycoprotein subunit gp120 of lentiviral particles, thereby forming Acryl-PEG-VP.

For example, the method can include concentrating viral particles using ultracentrifugation (e.g. at 25,000 rpm for 2 hours at 4° C.) and resuspending the particles in PBS (e.g., 25 mL). The suspension can then be combined with the polymer (e.g., 10 mg PEG-SVA dissolved in 25 mL PBS) providing for a combined solution (e.g., a combined solution of 50 mL volume). The mixture can then be shaken for a period of time at suitable temperature (e.g., overnight at 4° C.).

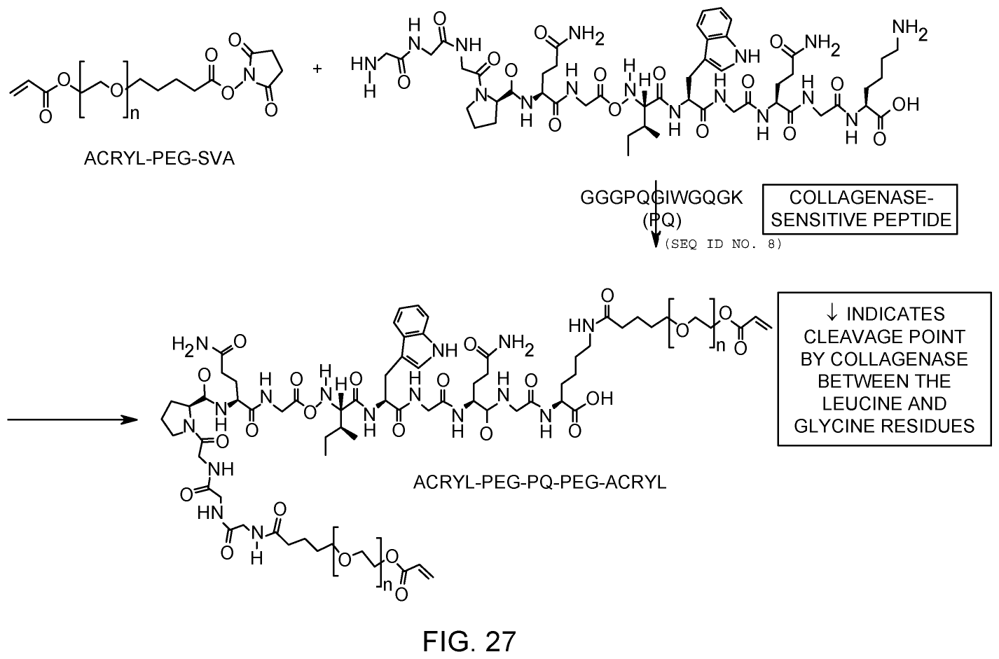

As further shown in FIG. 27, the Acryl-SVA can be rendered degradable. For example, a separate polymer mixture can be made to render PEG hydrogels degradable through covalent incorporation of the collagenase-sensitive peptide sequence, GGGPQG↓IWGQGK (SEQ ID NO:8), (PQ). PQ peptide reacts with Acryl-PEG-SVA to create Acryl-PEG-PQ-PEG-Acryl.

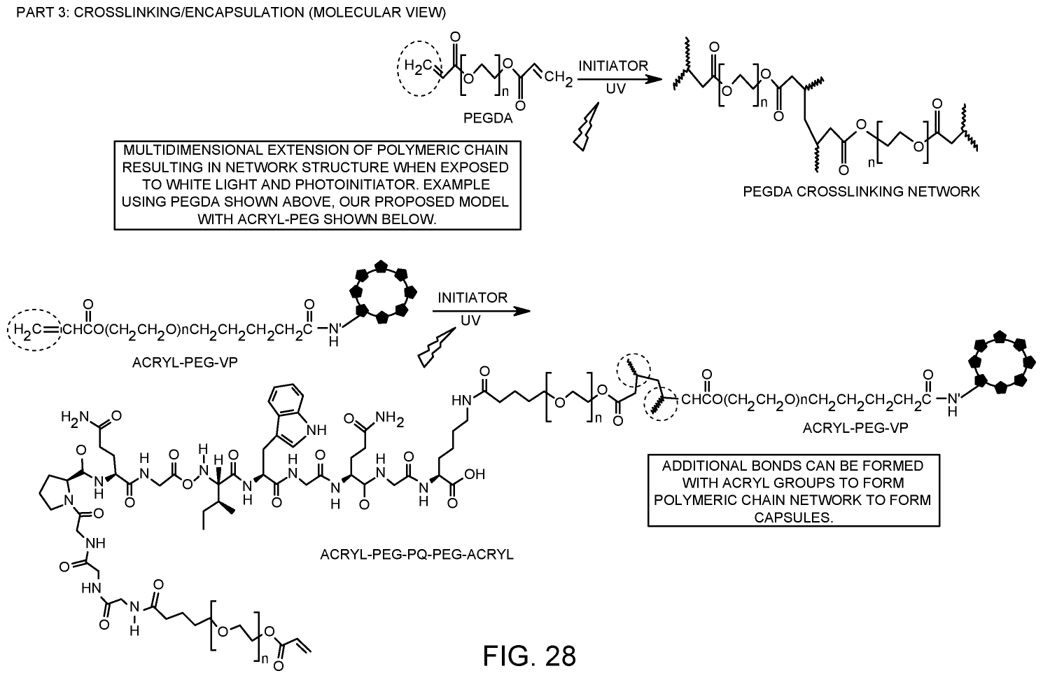

As further shown in FIG. 28, the crosslinking of polymers and encapsulation of cells can be accomplished using a dual photoinitiator emulsion-based technique, for example, as shown and described above with respect to FIG. 25. Exposure to white light and a photoinitiator can break Acryl group double bonds to form VP-PEG-Acryl-PEG-PQ-PEG-Acryl polymeric chains. Emulsion polymers begin forming when a free radical, acting as an initiator, breaks a double bond between two carbon atoms in an acrylic monomer, starting a reaction that can cause monomer units (e.g., as many as 10,000 monomer units) to bind together into a polymer chain.

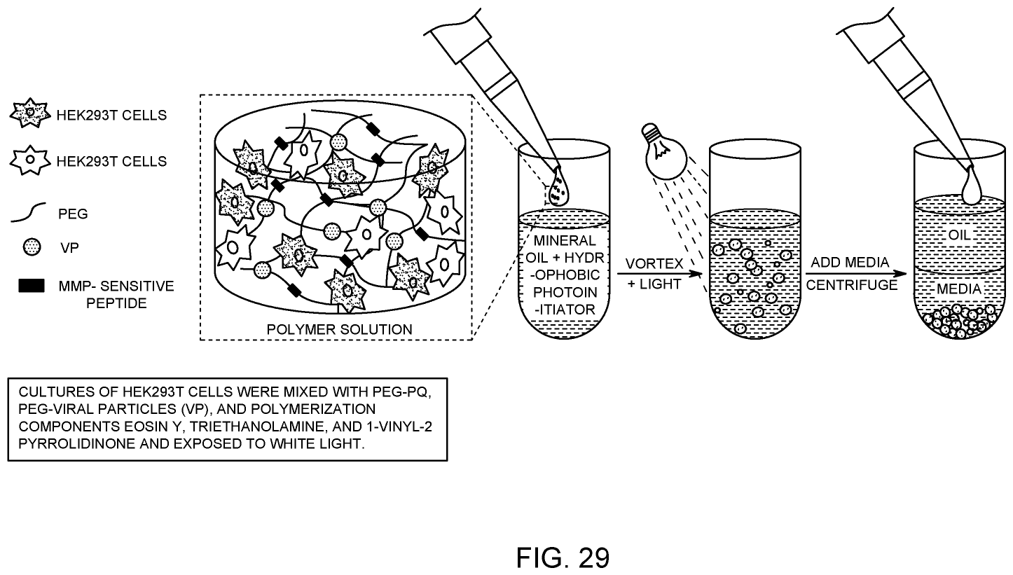

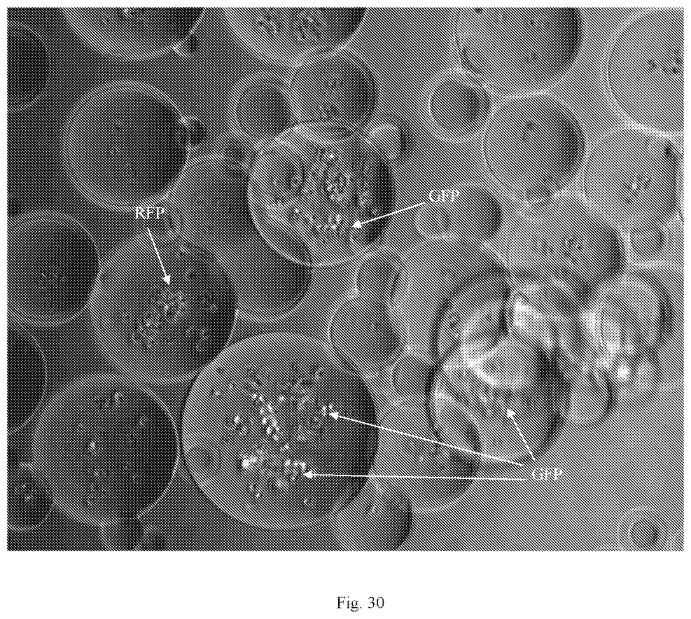

A bulk view of the process shown in FIG. 28 is shown in FIG. 29. As illustrated cell cultures (e.g., NK cells) can be mixed with PEG-PQ, PEG-VP, and polymerization components (e.g., eosin T, triethanolamine, and 1-vinyl-2 pyrrolidinone) and exposed to white light.

### EXEMPLIFICATION

### Example 1. Mechanical Properties of Polyethylene Glycol (PEG) Capsules

Current microcarriers are generally not mechanically durable and are known to sustain damage during bioreactor agitation processes, resulting in e.g. fragmented debris. Polyethylene glycol diacrylate (PEGDA) capsules were tested in preliminary studies under fast agitation conditions and were evaluated for visual integrity. FIGS. 3A-3C illustrate the mechanical durability of PEGDA capsules. PEGDA capsules were synthesized through a water-oil emulsification method and cultured in static (FIG. 3A), conventional speed (FIG. 3B), and high speed (FIG. 3C) agitation conditions. The photomicrographs of FIGS. 3A-3C show that the capsules maintain their integrity even at high rotations per minute (rpms) which are beyond the traditional speeds (˜75 rpm) used in cultures. FIGS. 3A-3C also show a lack of capsule fragment debris.

### Example 2. PEG Capsules Support Cell Growth

Preliminary studies with non-decorated PEGDA capsules were performed under static conditions to validate biomaterial properties. Polyethylene glycol (PEG) is a versatile polymer with tunable biochemical and mechanical properties and its safety profile is well-established. PEG offers a blank-state which, on its own, repels protein adsorption and subsequent cell-surface interactions, but also provides for the addition of bioactive ligands. As such, PEG provides an opportunity to create surfaces that promote cell interactions and adhesion while suppressing non-specific protein adsorption and cell adhesion.

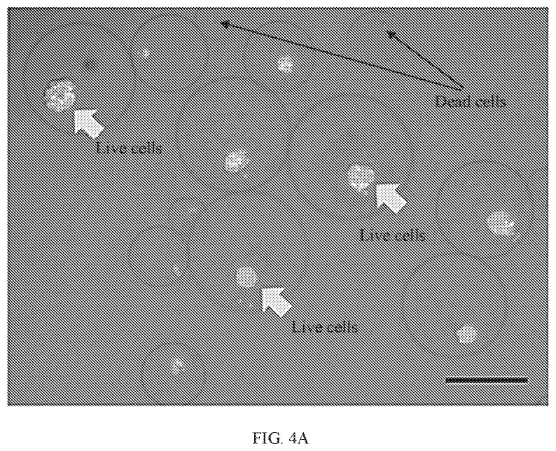

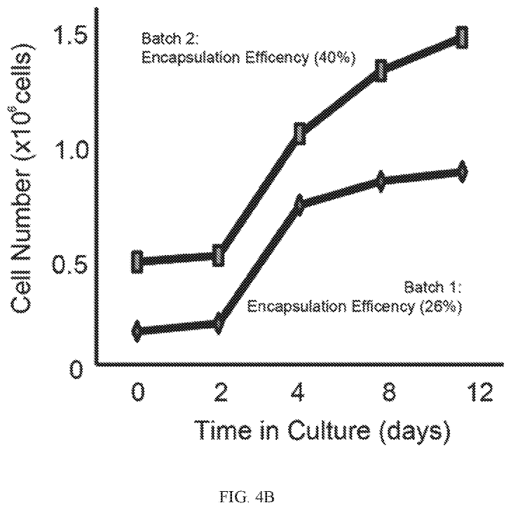

MSCs were encapsulated in PEGDA via white light polymerization. Cells were encapsulated at two different input cell concentrations to the cell-material bulk mixture. Viable cells detected by Calcein Acetoxymethyl (AM) staining were observed as MSC spheroids at the end of a 12-day culture process (FIG. 4A). Specifically, LIVE/DEAD™ Viability/Cytotoxicity Kit was used to detect live cells (green) with Calcein AM, and dead cells (red) with Ethidium Homodomer 1.

Encapsulation efficiency and cell proliferation were quantified over a 12 day culture process via Cell Titer 96® AQueous One Solution Cell Proliferation Assay. Results show an approximate 3-fold expansion in capsules over a 12-day period without inclusion of growth factors or adhesion peptides. Efficiencies of encapsulation correlated to the cell density per given feed material stock as expected. Encapsulated cells showed an approximate 2-3 day doubling time (within acceptable range for industrial MSC culture) without the addition of any growth factors or adhesion peptides to the capsule material (FIG. 4B).

### Example 3A. RGDS-Conjugated PEG Capsules Promotes Cell Attachment and Spreading

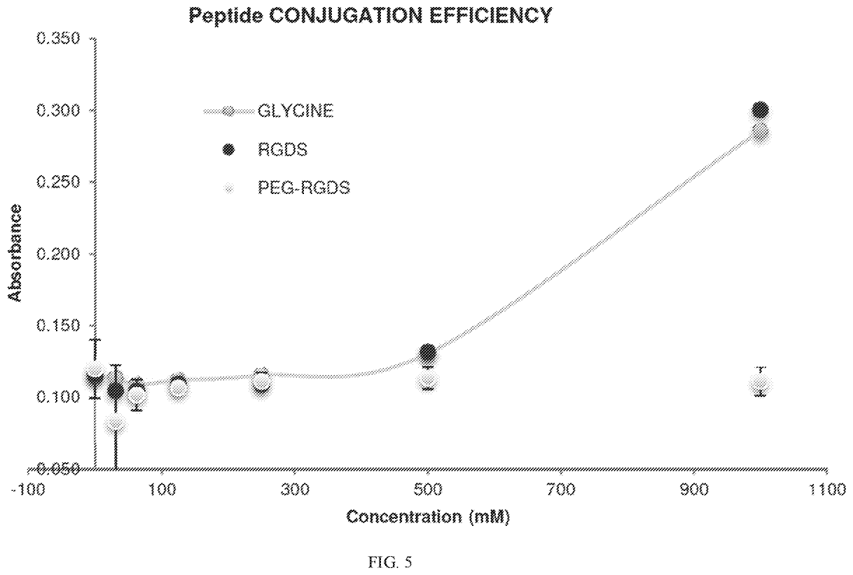

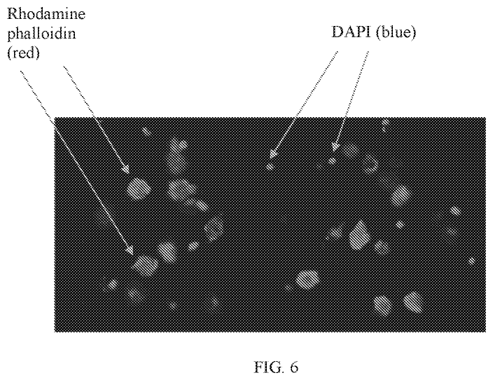

The peptide RGDS was successfully conjugated to PEG by reacting the peptide with acrylate-PEG-SVA. Adhesion peptide conjugation efficiency was assessed by detecting any unreacted free amines via a ninhydrin assay. Glycine was used as free-amine standard. PEG conjugated RGDS (PEG-RGDS) was assayed to detect any unreacted free amines. An 87% conjugation efficiency was achieved (FIG. 5). MSCs were encapsulated in a pilot study within adhesion peptide (RGDS) functionalized PEGDA capsules with a polymerized central core and cultured under dynamic conditions (125 rpm). It should be noted that the material of the shell and the core can be the same and the polymerization technique can either create a polymerized shell or a polymerized shell and core (which is referred to as a polymerized central core). The difference is in the degree of crosslinking. In liquid core capsules, the central core of the capsule is left uncrosslinked and only the shell is polymerized. In hydrogel or polymerized core capsules, the central core is also cross-linked. Cell-laden capsules sampled on day 3 were stained with rhodamine phalloidin and DAPI to visualize actin organization of the encapsulated cells (FIG. 6). Specifically, confocal images of rhodamine phalloidin (red) and DAPI (blue) stained encapsulated MSCs were sampled on day 3 from dynamic spinner flask cultures. The image of FIG. 6 demonstrates preliminary evidence of cell attachment and spreading.

### Example 3B. Effective Cell Attachment with Adhesive Capsules

Further testing was performed to assess the bioactivity of adhesives, the results of which are shown in FIGS. 14A-14I.

MSCs were encapsulated within adhesion peptide (RGDS) functionalized PEG capsules and cultured under dynamic conditions (125 rpm). PEG-RGDS at 5 mM (low) and 10 mM (high) concentration was used and cell spreading and attachment was assessed via confocal microscopy imaging of rhodamine phalloidin stained cells. No PEG-RGDS capsules were used as control. High cell spreading was demonstrated with 10 mM (high) adhesive on day 14 at the end of the culture process.

### Example 4A. Effect of Growth Factor in Cell Proliferation

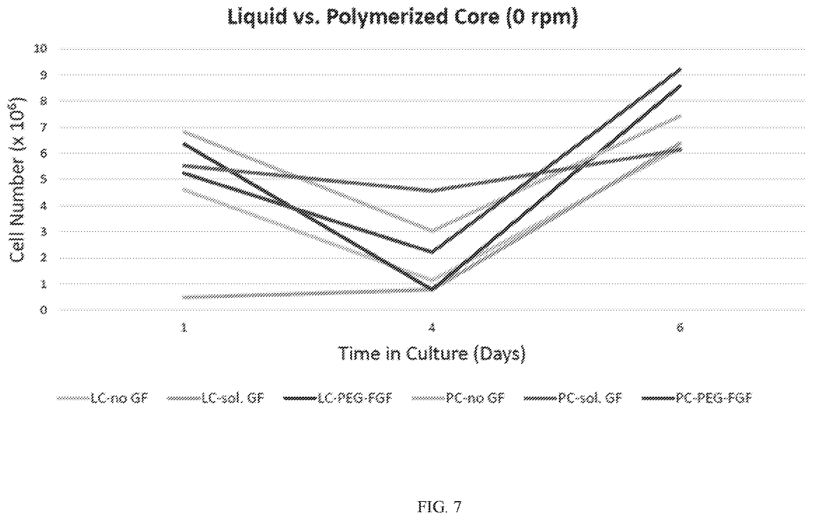

Cell-laden liquid core (LC) and polymerized core (PC) capsules were assessed under static cultures to study the effect of growth factors (GFs) on cell proliferation, the results of which are shown in FIG. 7. In particular, capsules were created with PEGylated RGDS and contained one of PEGylated FGF, soluble GF, or no GF. MSC were encapsulated in LC or PC capsules. Samples were taken on days 1, 4, and 6 to assess cell proliferation. The results show capsules including PEG-FGF, whether the PEG-FGF was located in a hydrogel core (i.e., PC capsules) or in a shell of the capsule (i.e., LC capsule) had the greatest cell proliferation while soluble FGF in PC capsules maintained cell numbers at the encapsulated seeding density.

### Example 4B. Addition of Growth Factor in Material Improves Post-Encapsulation Viability

Further testing was performed to assess MSC viability in capsules with and without FGF. Varying concentrations of PEGylated adhesive peptide RGDS and PEGylated FGF peptide were tested, the results of which are shown in FIGS. 15A-15F.

MSCs were encapsulated within adhesion peptide (RGDS) and FGF functionalized PEG capsules and cultured under dynamic conditions (125 rpm). PEGylated FGF was added at 2.5 μg/L for each 0 mM, 5 mM, and 10 mM PEG-RGDS samples. Comparison against no FGF controls demonstrates higher post-encapsulation viability in the presence of PEGylated FGF.

Capsules with FGF peptide showed higher day 1 post-encapsulation viability than capsules lacking FGF.

### Example 5A. Effect of Agitation on Cell Viability, Proliferation, and Carrier Integrity

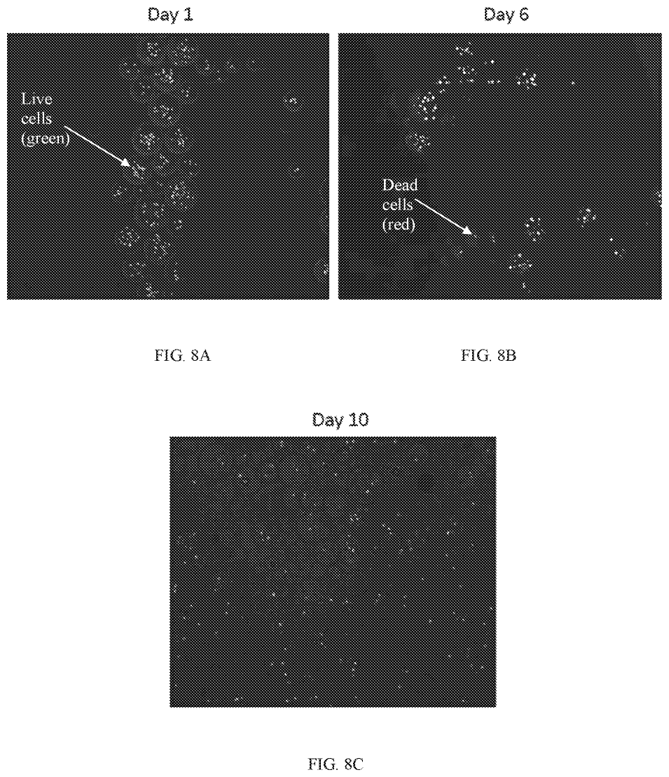

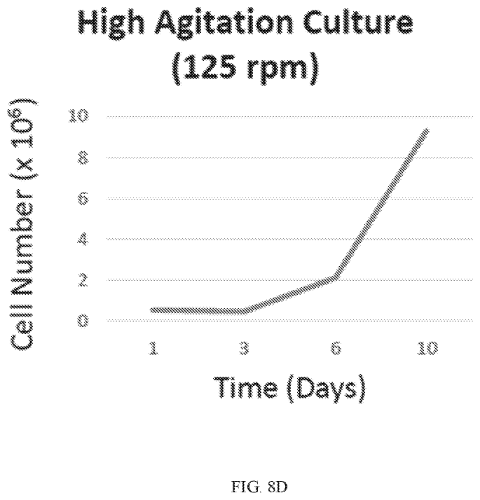

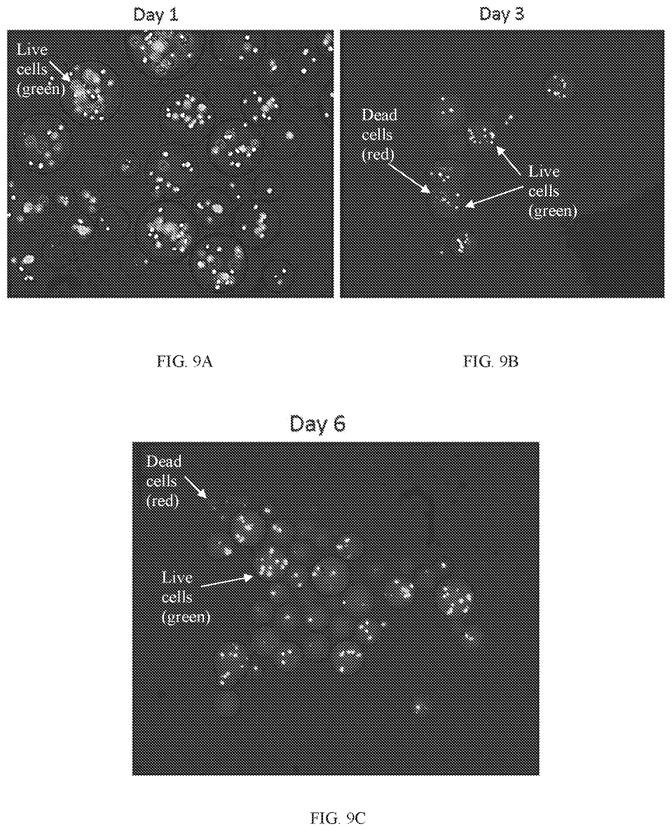

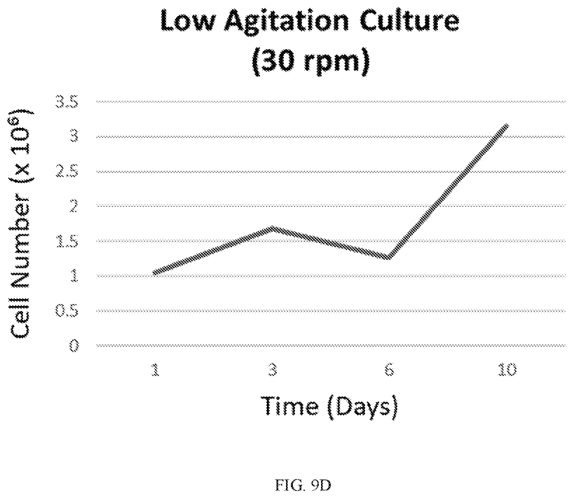

Cell-laden polymerized core capsules with PEGylated RGDS and FGF were exposed to high (125 rpm) and low (30 rpm) agitation speeds in spinner flasks. The results are shown in FIGS. 8A-8D (high agitation culture) and 9A-9D (low agitation culture). Samples were taken on days 1, 3, 6 and 10. High agitation cultures resulted in an approximate ˜18 fold increase in encapsulated cell number while low agitation cultures resulted in only a 3 fold increase in cell number at the end of the culture period. This might be attributed to inefficient nutrient and waste exchange at low agitation while also highlighting that high agitation/shear is not detrimental cells encapsulated within PEG capsules.

### Example 5B. MSC Viability after High-Speed Agitation Culture

Further testing was performed to assess MSC viability after high-speed agitation culture (125 rpm) in spinner flasks in the presence of bioactive factors PEG-RGDS and PEG-FGF in degradable PEG capsules.

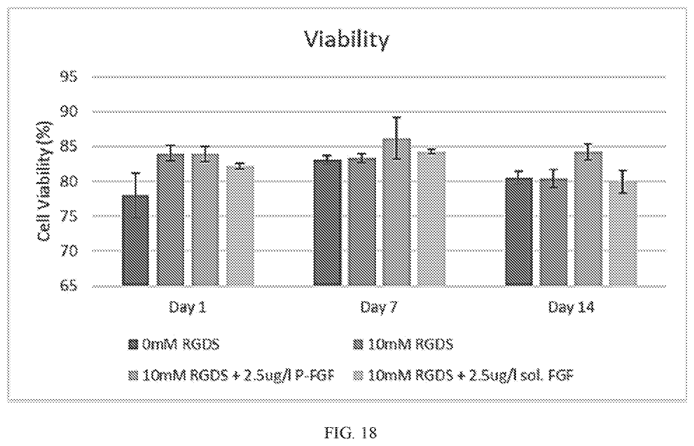

The results are shown in FIG. 18. The presence of both bioactive components, PEG-RGDS and PEG-FGF provided improved cell viability and significantly improved viability over soluble FGF.

### Example 6. Properties of Polymerized Core (PC) Capsules

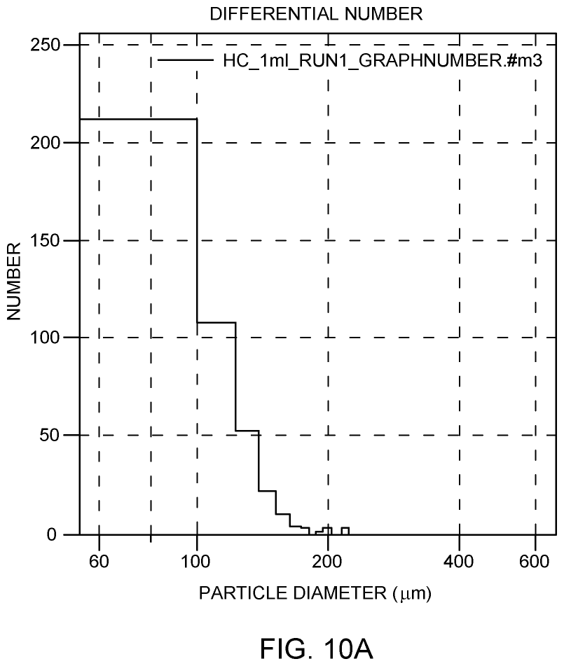

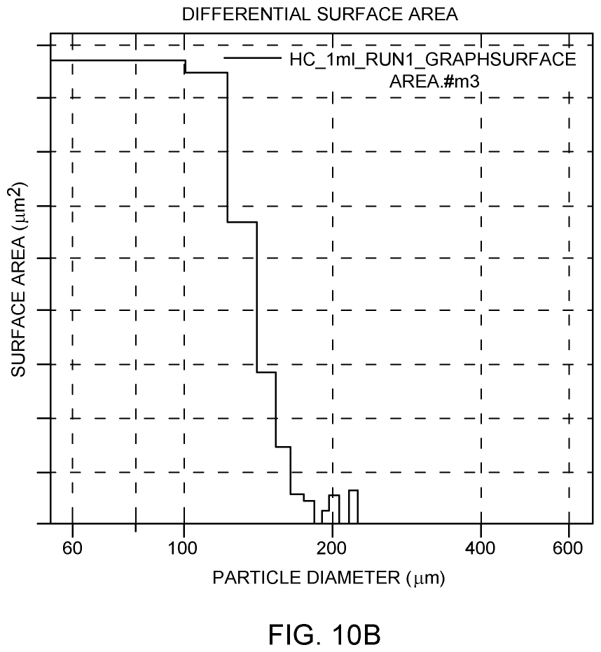

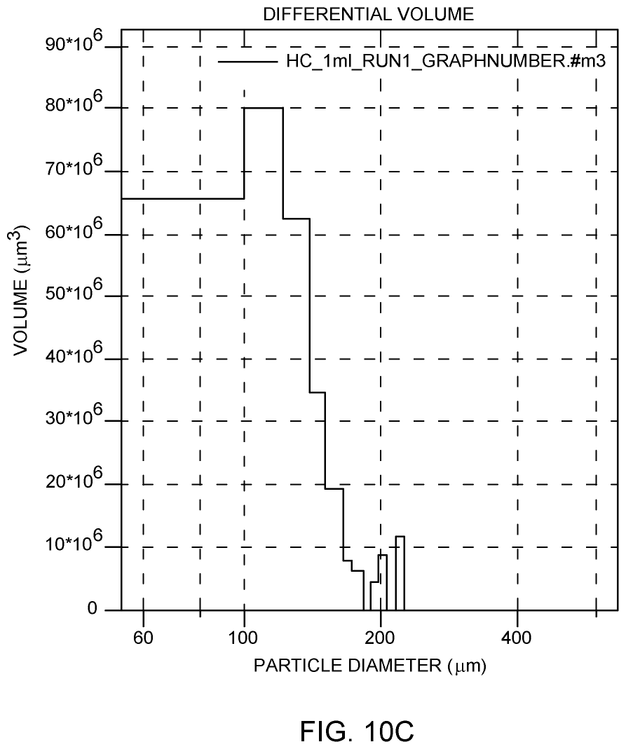

PC capsules were analyzed on a Coulter Counter to study carrier size distribution, surface area, and volume. Empty PEGDA capsules were produced as a polymerized core via photo-polymerization without adhesion peptides or GFs. Capsules of heterogeneous size distribution were produced within acceptable and expected diameter limits of 70 um-250 um. Average volume and surface area of the thus produced capsules were 3×10⁷ um³ and 1.5×10⁶ um², respectively for PC and 7.2×10⁶ um³ and 3.4×10⁵ um², respectively for LC. The results are shown in FIGS. 10A-10C.

### Example 7. Properties of Liquid Core (LC) Capsules

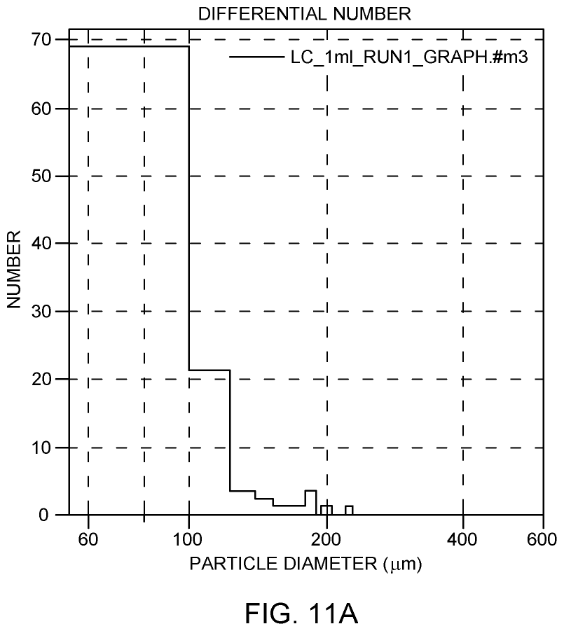

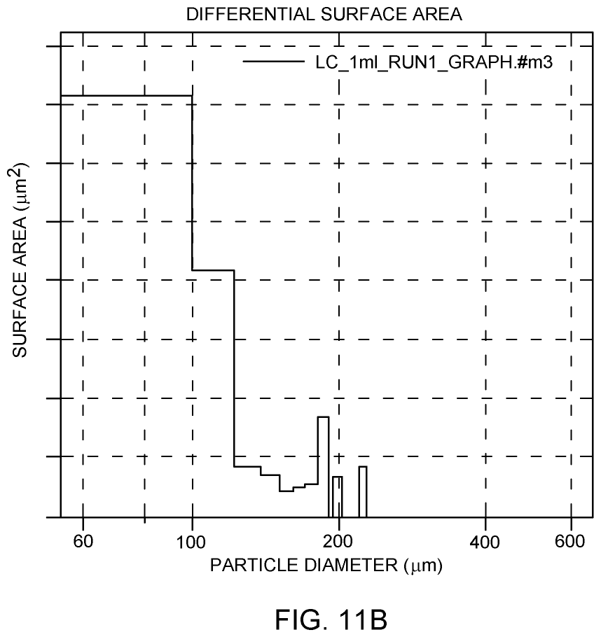

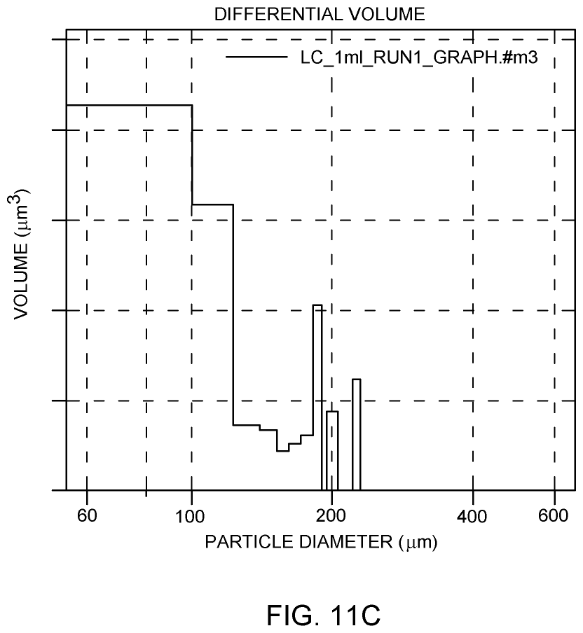

LC capsules were analyzed on a Coulter Counter to study carrier size distribution, surface area, and volume. The LC capsules were formed with a liquid core via photo-polymerization without adhesion peptides or GFs. Capsules of heterogeneous size distribution were produced within acceptable and expected diameter limits of 70 um-250 um. Average volume and surface area of the thus produced capsules were 3×107 um3 and 1.5×106 um2, respectively for PC and 7.2×106 um3 and 3.4×105 um2, respectively for LC. The results are shown in FIGS. 11A-11C.

### Example 8. Capsule Degradation

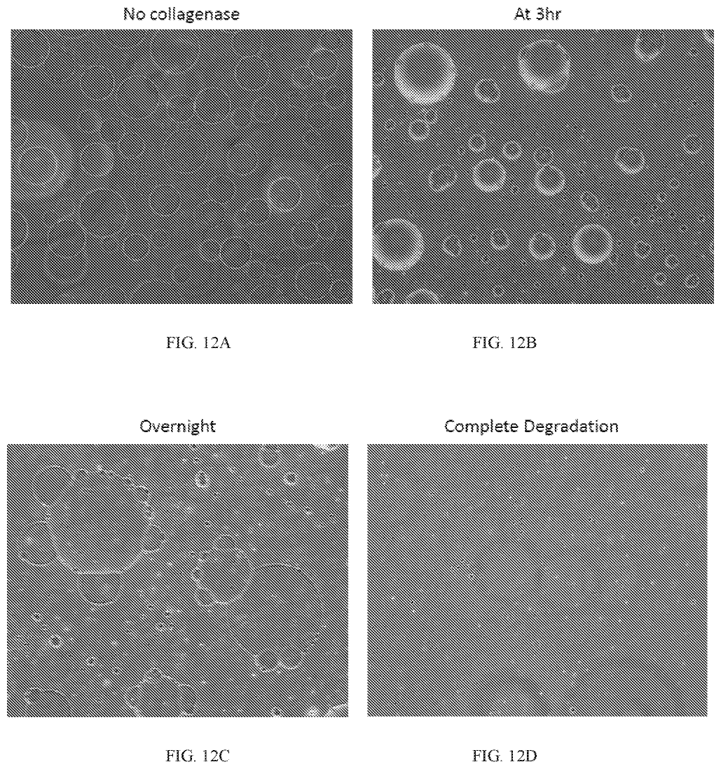

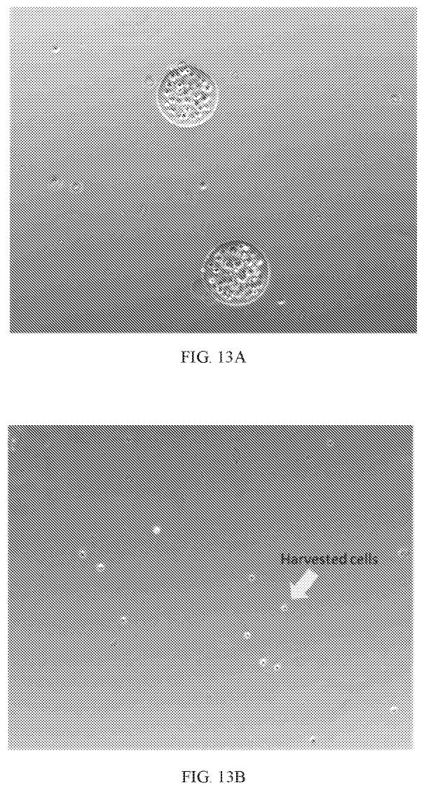

Collagenase degradable peptide (GGGPQG↓IWGQGK) (SEQ ID NO:8) was conjugated to PEG to form degradable capsules. Collagenase degradable peptide was conjugated in a 1:2 (peptide:PEG) molar ratio. The conjugated PEG was then used to form non-cell-laden (FIGS. 12A-12D) and cell-laden (FIGS. 13A-13B) PC capsules via white light polymerization. No adhesion peptides or GFs were used. The degradable capsules were then exposed to collagenase and capsule integrity was analyzed at different time points under static conditions (FIGS. 12A-12D) as well as under mechanical agitation conditions (FIGS. 13A-13B). The results are shown in FIGS. 12A-12D and 13A-13B. As shown in FIG. 12A, capsules having the protease-sensitive peptide and that were not incubated with collagenase were not degraded. As shown in FIG. 12B-D, the capsules doped with the protease-sensitive peptide show signs of degradation over time upon exposure to collagenase. The degradation without mechanical agitation is a process that can take hours, in this example, >24 hours.

As shown in FIGS. 13A-13B, the degradation of the cell-laden PC capsules occurred almost instantaneously after mechanical agitation, which, in this example, was performed by repeated extrusion of the solution. As shown in both FIGS. 12D and 13B, no remaining subparticles over a size of about 1 μm in approximation were found. In the example of cell-laden capsules, the arrow of FIG. 13B points to harvested cells that remain after capsule degradation.

### Example 9. MSC Morphology Maintained after Cell Purification

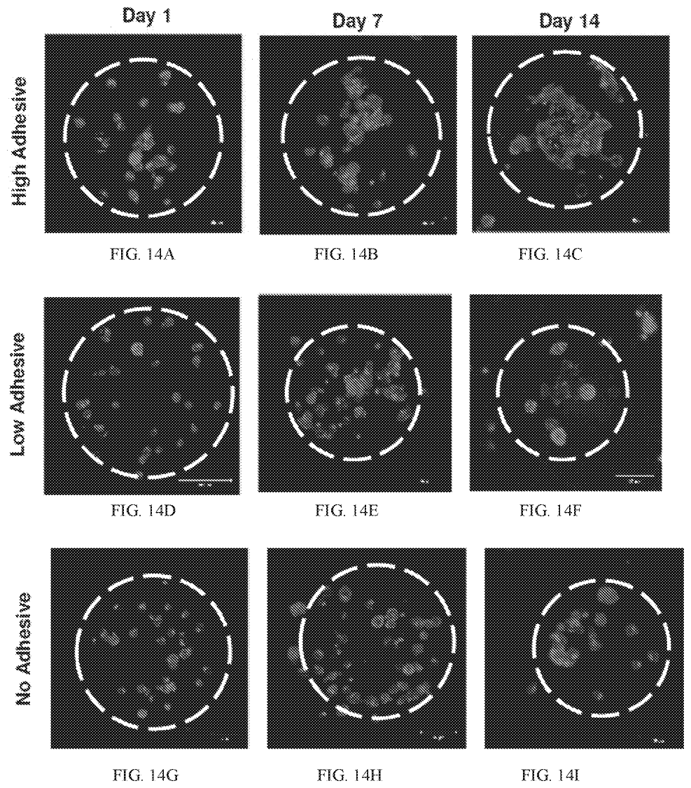

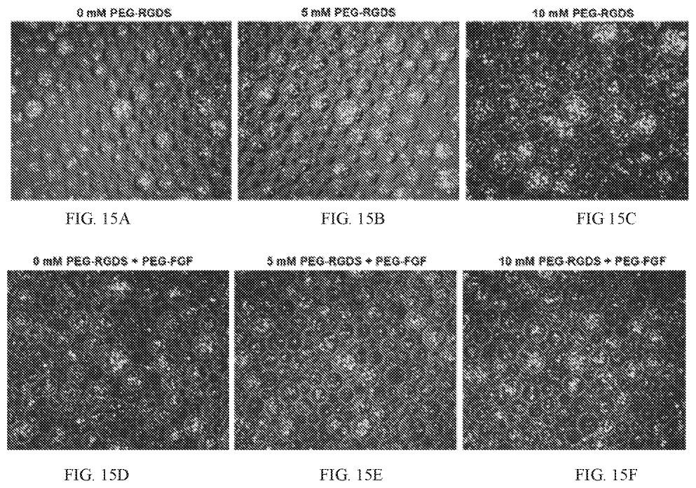

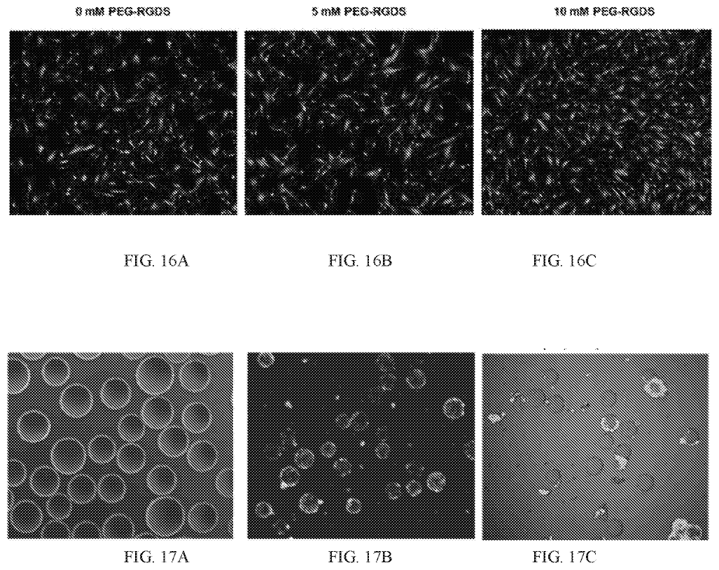

Further testing was performed to assess MSC morphology following harvesting from capsules that included varying concentrations of adhesive peptides, the results of which are shown in FIGS. 16A-16C. These tests were performed to confirm that the capsule materials (PEG, PQ or RGDS) did not have any detrimental effect on TCP attachment, spreading and proliferation of MSCs post-harvest. No difference in cell morphology was observed between the samples. PEG-RGDS at 10 mM demonstrated a higher cell yield.

### Example 10. Comparison to Industry-Standard Carriers

Microcarriers were equilibrated in cell culture media, and MSC cells were allowed to adhere to the microcarrier surface under static conditions (FIG. 17A-B). Cytodex 3 (Corning) microcarriers were used. Microcarrier-adhered MSCs were cultured at 70 rpm under standard cell culture media conditions.

After forty-eight hours in dynamic culture, MSCs detached from the microcarrier surface and aggregate cell debris was visible in the spinner flask, as shown in FIG. 17C.

### Example 11. Comparative Viability of MSCs in PEGDA with 0 mM or 10 mM RGDS

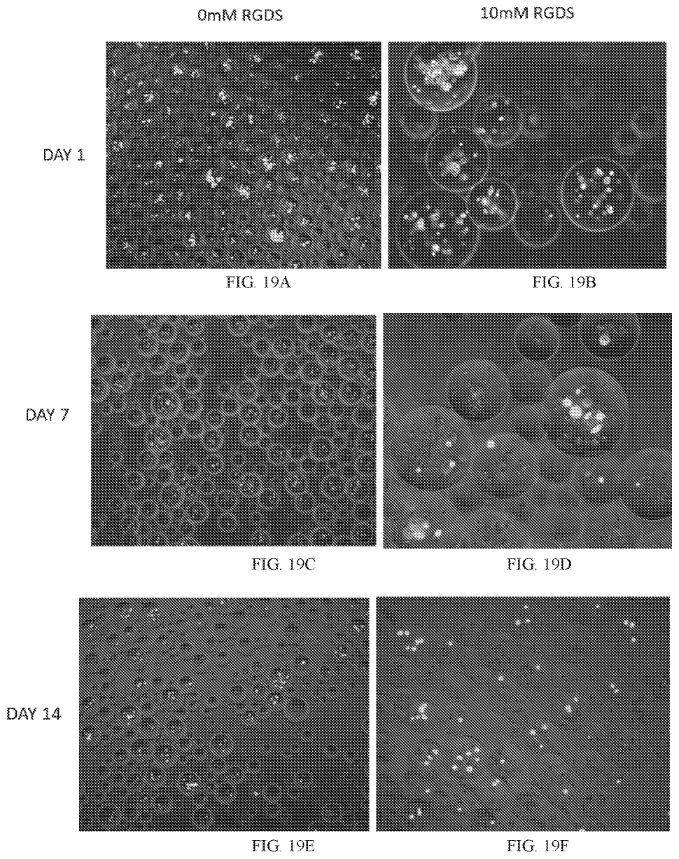

Testing was performed to assess the viability of MSCs in PEGDA with and without 10 mM conjugated RGDS. Samples were taken at Days 1, 7, and 14, the results of which are shown FIGS. 19A-19F. The purpose of this experiment was to determine if adhesive peptides effect the viability and proliferation of encapsulated MSCs. Encapsulated MSCs were cultured in spinner flasks at high agitation (125 rpm) and maintained in an incubator at 37° C./5% CO₂. In the presence of a bioactive component, PEG-RGDS provided improved cell viability by Day 1, over the 0 mM PEG-RGDS cohort.

### Example 12. Comparative Viability of MSCs in PEGDA with 10 mM RGDS and Varying Concentrations of bFGF

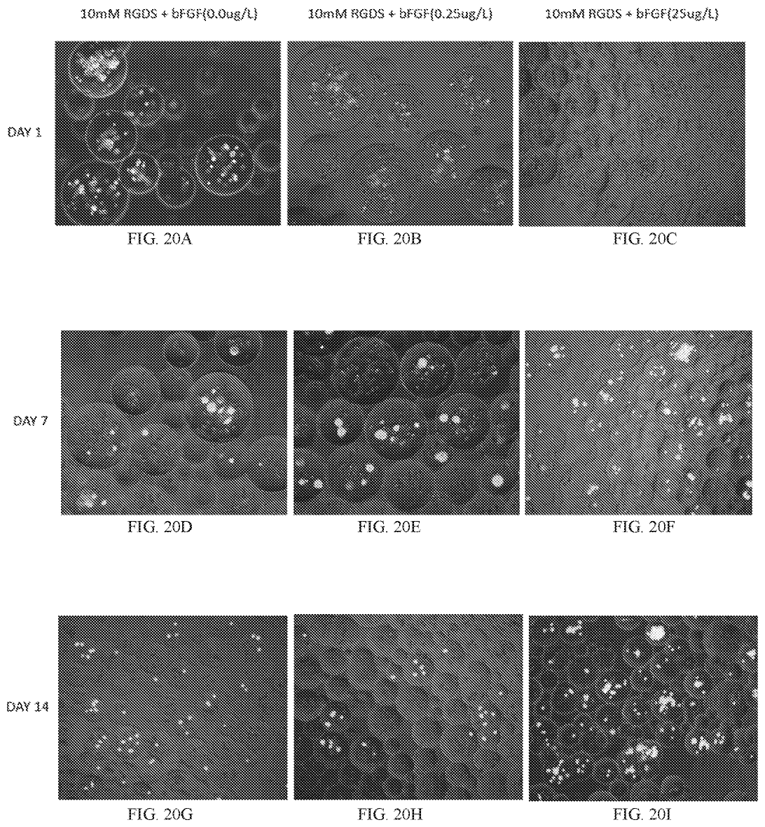

Testing was performed to assess the viability of MSCs in PEGDA with varying concentrations of bFGF (0 μg/L, 0.25 μg/L, and 25 μg/L). Samples were taken at days 1, 7, and 14, the results of which are shown FIGS. 20A-20I. Encapsulated MSCs were cultured in spinner flasks at high agitation (125 rpm) and maintained in an incubator at 37° C./5% CO₂. MSC proliferation was positively affected by the concentration of bFGF. With higher concentrations of bFGF conjugated to the microcapsules, we have found denser pockets along the microcapsule wall of proliferating MSCs. As seen in FIGS. 20A-20I, the presence of both bioactive components, PEG-RGDS and PEG-FGF, provided improved cell viability and significantly improved viability over soluble FGF.

### Example 13. Comparative Viability of MSCs in Non-Degradable and Degradable Capsules

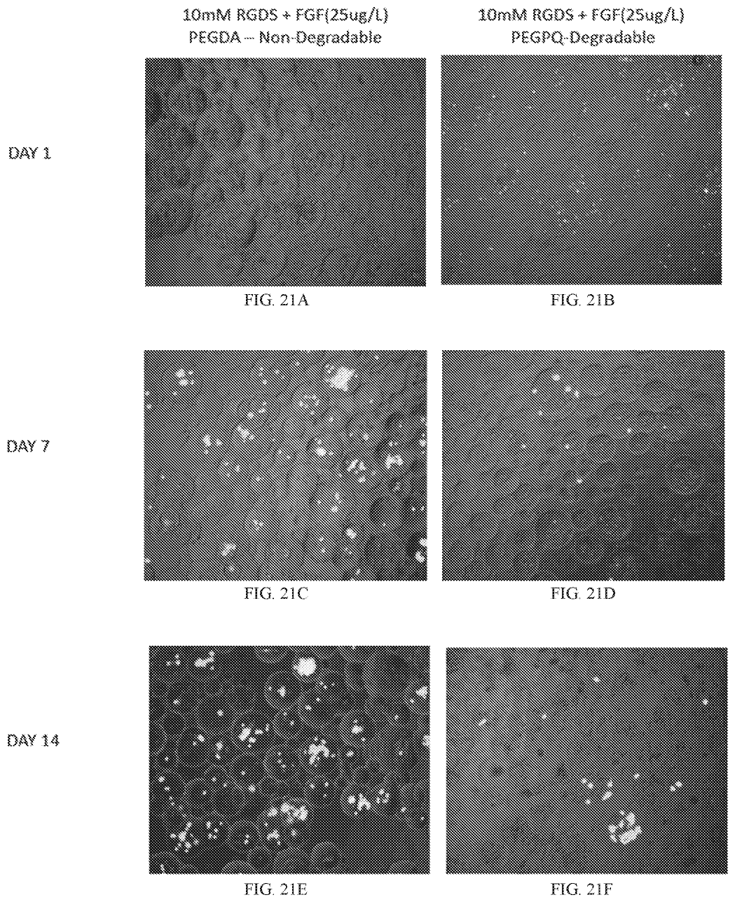

Testing was performed to assess the viability of MSCs in non-degradable and degradable capsules. Capsules including 10 mM RGDS and 25 FGF were created with PEGDA (non-degradable) and PEGPQ (degradable). Samples were taken at Days 1, 7, and 14, the results of which are shown FIGS. 21A-21F.

Encapsulated MSCs were cultured in spinner flasks at high agitation (125 rpm) and maintained in an incubator at 37° C./5% CO₂. MSCs in PEGDA (non-degradable) capsules supplemented with bFGF (25 μg/L) showed dense viable pockets along the microcapsule wall, suggestive of MSC proliferation. MSCs in PEGPQ (degradable) capsules produced dense viable pockets, however, were fewer in number compared to MSCs encapsulated in PEGDA. PQ peptide may be producing an acidic microenvironment within the capsule, which may negatively affect MSC viability.

### Example 14. Comparative Viability of Multiple Cell Types in PEGDA

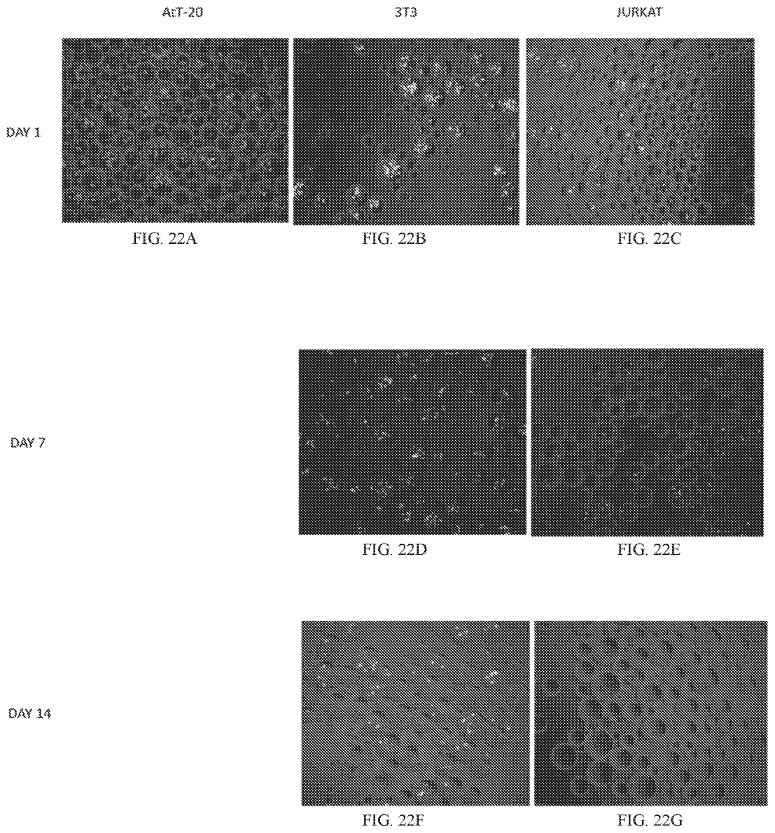

Testing was performed with multiple cell lineages, including AtT-20, 3T3, and JURKAT cells. The cells were encapsulated in synthesized PEGDA capsules with 0 mM and sampled at days 1, 7, and 14, the results of which are shown in FIGS. 22A-22G.

Encapsulated MSCs were cultured in spinner flasks at high agitation (125 rpm) and maintained in an incubator at 37° C./5% CO₂. The purpose of this experiment was to determine if PEGDA capsules are not only compatible with MSCs, but also with additional cell lines that are relevant to the development of cell therapy products. We have found PEGDA capsules are indeed compatible with adherent cell lines that include AtT-20's and 3T3's, as opposed to Jurkat cell lines grown in suspension. Photos for AtT-20 at Days 7 and 14 were not provided as these studies are ongoing.

### Example 15. PBMC Stimulation

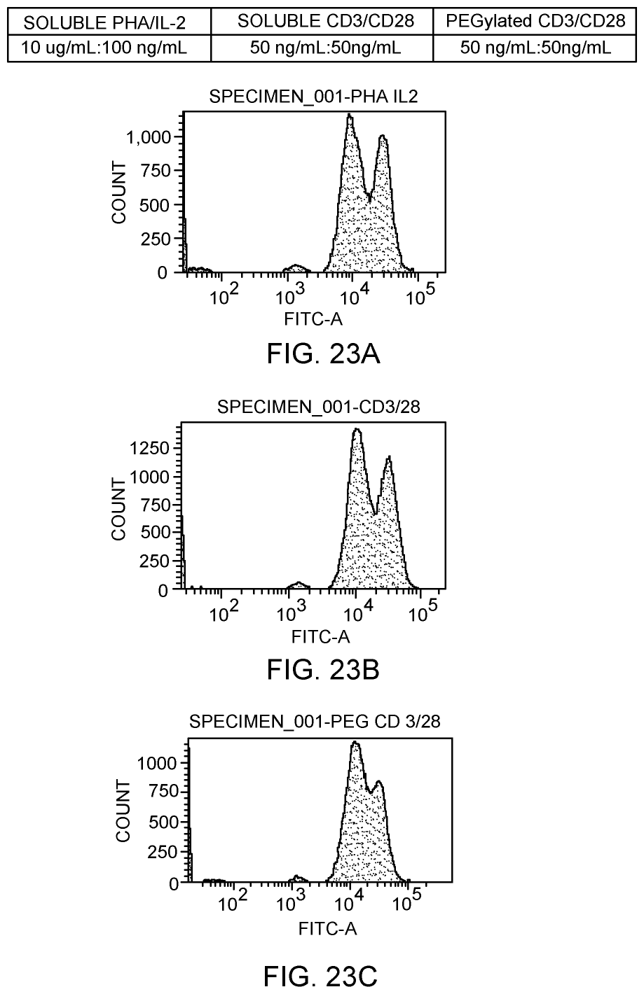

Testing was performed to compare the efficiency of PBMC stimulation with soluble CD3/CD28 vs PEGylated CD3/CD28, the results of which are shown in FIGS. 23A-23C. CFSE stained cells were encapsulated in PEGPQ capsules containing soluble PHA/IL2 or soluble CD3/CD28 or PEGylated CD3/CD28. Cells were harvested on day 4 post-stimulation, and CFSE signal was assessed using flow cytometry analysis. No difference in stimulation efficiency was observed between soluble and PEGylated CD3/CD28. PBMC activation was also achieved with PHA/IL2 in the capsule suggesting that stimulation efficiency is not lost during the encapsulation process or in the capsules compare to suspended cells.

### Example 16. Viral Particles

Testing was performed in which Lentivirus (LV) particles were conjugated and encapsulated with HEK 293T cells by the process described above with respect to FIGS. 25-29. Results are shown in FIG. 30. RFP (Red fluorescent protein) is the gene construct that is carried by the lentivirus particle (e.g., an expression marker to assess transduction efficiency based on integration of the RFP gene into the host cell HEK293T genome). RFP in this image corresponds to the fluorophore that fluoresces red when excited, such as when the gene is in the cell of interest, which can confirm the occurrence of lentiviral transduction of HEK293T cells with the gene of interest. GFP (green fluorescent protein) in the image corresponds to live cells. (GFP from Calcein AM staining) is a dye used to determine cell viability because it readily permeates intact, live cells. In particular, 2 μL of Calcein was added to 1 mL of media and encapsulated cells prior to imaging using a ZEISS microscope upon which the image of FIG. 30 was collected. In this particular experiment, we were testing the proof of concept that conjugated lentiviral particles could transduce target cells with a gene of interest (RFP) and that, after a period of time, capsules will still contain live cells (GFP stain). We concluded the ability to encapsulate cells and conjugate lentiviral particles successfully inside of PEG capsules, while maintaining the infectivity of the particles by showing successful transduction of encapsulated HEK293T cells with lentivirus using the RFP marker.

### REFERENCES

1. Meignier, B., H. Mougeot, and H. Favre, Foot and mouth disease virus production on microcarrier-grown cells. Vol. 46. 1980. 249-56.
2. Genzel, Y., et al., Wave microcarrier cultivation of MDCK cells for influenza virus production in serum containing and serum-free media. Vaccine, 2006. 24(35): p. 6074-6087.
3. Wolfe, M., et al., Isolation and Culture of Bone Marrow-Derived Human Multipotent Stromal Cells (hMSCs), in Mesenchymal Stem Cells: Methods and Protocols, D. J. Prockop, B. A. Bunnell, and D. G. Phinney, Editors. 2008, Humana Press: Totowa, N.J. p. 3-25.
4. Hanley, P. J., et al., Manufacturing mesenchymal stromal cells for phase I clinical trials. Cytotherapy, 2013. 15(4): p. 416-422.
5. Rojewski, M. T., et al., GMP-Compliant Isolation and Expansion of Bone Marrow-Derived MSCs in the Closed, Automated Device Quantum Cell Expansion System. Cell Transplantation, 2013. 22(11): p. 1981-2000.
6. Jones, M., et al., Genetic stability of bone marrow-derived human mesenchymal stromal cells in the Quantum System. Cytotherapy, 2013. 15(11): p. 1323-1339.
7. dos Santos, F., et al., Ex Vivo Expansion of Human Mesenchymal Stem Cells on Microcarriers, in Mesenchymal Stem Cell Assays and Applications, M. Vemuri, L. G. Chase, and M. S. Rao, Editors. 2011, Humana Press: Totowa, N.J. p. 189-198.
8. Hewitt, C. J., et al., Expansion of human mesenchymal stem cells on microcarriers. Biotechnology Letters, 2011. 33(11): p. 2325.
9. Rafiq, Q. A., et al., Culture of human mesenchymal stem cells on microcarriers in a 5 l stirred-tank bioreactor. Biotechnology Letters, 2013. 35(8): p. 1233-1245.
10. Simaria, A. S., et al., Allogeneic Cell Therapy Bioprocess Economics and Optimization: Single-Use Cell Expansion Technologies. Biotechnology and Bioengineering, 2014. 111(1): p. 69-83.
11. Schnitzler, A. C., et al., Bioprocessing of human mesenchymal stem/stromal cells for therapeutic use: Current technologies and challenges. Biochemical Engineering Journal, 2016. 108: p. 3-13.
12. Rafiq, Q. A., K. Coopman, and C. J. Hewitt, Scale-up of human mesenchymal stem cell culture: current technologies and future challenges. Current Opinion in Chemical Engineering, 2013. 2(1): p. 8-16.
13. Nienow, A. W., Reactor Engineering in Large Scale Animal Cell Culture. Cytotechnology, 2006. 50(1-3): p. 9-33.
14. Jung, S., et al., Large-scale production of human mesenchymal stem cells for clinical applications. Vol. 59. 2012. 106-20.
15. Nienow, A. W., et al., A potentially scalable method for the harvesting of hMSCs from microcarriers. Biochemical Engineering Journal, 2014. 85 (Supplement C): p. 79-88.
16. Rafiq, Q. and C. Hewitt, Cell therapies: why scale matters. Vol. 3. 2015. 97-99.
17. Tan, K. Y., S. Reuveny, and S. K. W. Oh, Recent advances in serum-free microcarrier expansion of mesenchymal stromal cells: Parameters to be optimized. Biochemical and Biophysical Research Communications, 2016. 473(3): p. 769-773.
18. dos Santos, F., et al., Toward a Clinical-Grade Expansion of Mesenchymal Stem Cells from Human Sources: A Microcarrier-Based Culture System Under Xeno-Free Conditions. Tissue Engineering. Part C, Methods, 2011. 17(12): p. 1201-1210.
19. Yang, H. S., et al., Suspension culture of mammalian cells using thermosensitive microcarrier that allows cell detachment without proteolytic enzyme treatment. Cell Transplant, 2010. 19(9): p. 1123-32.
20. Shekaran, A., et al., Biodegradable ECM-coated PCL microcarriers support scalable human early MSC expansion and in vivo bone formation. Cytotherapy, 2016. 18(10): p. 1332-44.
21. Rafiq, Q. A., et al., Culture of human mesenchymal stem cells on microcarriers in a 5 l stirred-tank bioreactor. Biotechnol Lett, 2013. 35(8): p. 1233-45.
22. Rowley, J., et al., Meeting Lot-Size Challenges of Manufacturing Adherent Cells for Therapy. BioProcess International, 2012. 10(3): p. 16-22.
23. Heathman, T. R., et al., Expansion, harvest and cryopreservation of human mesenchymal stem cells in a serum-free microcarrier process. Biotechnology and bioengineering, 2015. 112(8): p. 1696-1707.
24. Pal, R., M. Hanwate, and S. M. Totey, Effect of holding time, temperature and different parenteral solutions on viability and functionality of adult bone marrow-derived mesenchymal stem cells before transplantation. J Tissue Eng Regen Med, 2008. 2(7): p. 436-44.
25. Nienow, A. W., et al., A potentially scalable method for the harvesting of hMSCs from microcarriers. Biochemical Engineering Journal, 2014. 85: p. 79-88.
26. Nienow, A. W., et al., Agitation conditions for the culture and detachment of hMSCs from microcarriers in multiple bioreactor platforms. Biochemical Engineering Journal, 2016. 108: p. 24-29.
27. Masri, M. F., et al., An ultra scale-down methodology to characterize aspects of the response of human cells to processing by membrane separation operations. Biotechnology and Bioengineering, 2017: p. n/a-n/a.
28. Kirouac, D. C. and P. W. Zandstra, The systematic production of cells for cell therapies. Cell Stem Cell, 2008. 3(4): p. 369-81.
29. Stathopoulos, N. A. and J. D. Hellums, Shear stress effects on human embryonic kidney cells in Vitro. Biotechnology and Bioengineering, 1985. 27(7): p. 1021-1026.
30. Croughan, M. S., J.-F. P. Hamel, and D. I. C. Wang, Effects of microcarrier concentration in animal cell culture. Biotechnology and Bioengineering, 1988. 32(8): p. 975-982.
31. Ma, N., et al., Quantitative Studies of Cell-Bubble Interactions and Cell Damage at Different Pluronic F-68 and Cell Concentrations. Biotechnology Progress, 2004. 20(4): p. 1183-1191.
32. Chen, A. K.-L., S. Reuveny, and S. K. W. Oh, Application of human mesenchymal and pluripotent stem cell microcarrier cultures in cellular therapy: Achievements and future direction. Biotechnology Advances, 2013. 31(7): p. 1032-1046.
33. Moran, E., A microcarrier-based cell culture process for the production of a bovine respiratory syncytial virus vaccine. Cytotechnology, 1999. 29(2): p. 135-149.
34. Ng, Y. C., J. M. Berry, and M. Butler, Optimization of physical parameters for cell attachment and growth on macroporous microcarriers. Biotechnology and Bioengineering, 1996. 50(6): p. 627-635.
35. Gupta, P., et al., Optimization of agitation speed in spinner flask for microcarrier structural integrity and expansion of induced pluripotent stem cells. Cytotechnology, 2016. 68(1): p. 45-59.
36. Rokstad, A. M. A., et al., Advances in biocompatibility and physico-chemical characterization of microspheres for cell encapsulation. Advanced Drug Delivery Reviews, 2014. 67-68: p. 111-130.
37. Qi, M., Transplantation of Encapsulated Pancreatic Islets as a Treatment for Patients with Type 1 Diabetes Mellitus. Advances in Medicine, 2014.
38. Desai, T. S., L. D., Advances in islet encapsulation technologies. Nat. Rev. Drug Discov., 2016.
39. Scharp, D. W. and P. Marchetti, Encapsulated islets for diabetes therapy: History, current progress, and critical issues requiring solution. Advanced Drug Delivery Reviews, 2014. 67-68: p. 35-73.
40. Soon-Shiong, P., et al., Insulin independence in a type 1 diabetic patient after encapsulated islet transplantation. The Lancet, 1994. 343(8903): p. 950-951.
41. Calafiore, R., et al., Microencapsulated pancreatic islet allografts into nonimmunosuppressed patients with type 1 diabetes. Diabetes care, 2006. 29(1): p. 137-138.
42. Narang, A. S. and R. I. Mahato, Biological and biomaterial approaches for improved islet transplantation. Pharmacological reviews, 2006. 58(2): p. 194-243.
43. Duvivier-Kali, V. F., et al., Complete protection of islets against allorejection and autoimmunity by a simple barium-alginate membrane. Diabetes, 2001. 50(8): p. 1698-1705.
44. Van Raamsdonk, J., et al., Deterioration of polyamino acid-coated alginate microcapsules in vivo. Journal of Biomaterials Science, Polymer Edition, 2002. 13(8): p. 863-884.
45. Omer, A., et al., Long-term normoglycemia in rats receiving transplants with encapsulated islets. Transplantation, 2005. 79(1): p. 52-58.
46. Tuch, B. E., et al., Safety and viability of microencapsulated human islets transplanted into diabetic humans. Diabetes care, 2009. 32(10): p. 1887-1889.
47. Vaithilingam, V., et al., Effect of prolonged gelling time on the intrinsic properties of barium alginate microcapsules and its biocompatibility. Journal of microencapsulation, 2011. 28(6): p. 499-507.
48. Vegas, A. J., et al., Combinatorial hydrogel library enables identification of materials that mitigate the foreign body response in primates. Nature biotechnology, 2016. 34(3): p. 345-352.
49. Vegas, A. J., et al., Long-term glycemic control using polymer-encapsulated human stem cell-derived beta cells in immune-competent mice. Nature medicine, 2016. 22(3): p. 306-311.
50. Poncelet, D., et al., Production of alginate beads by emulsification/internal gelation. I. Methodology. Applied Microbiology and Biotechnology, 1992. 38(1): p. 39-45.
51. Hoesli, C. A., et al., Pancreatic cell immobilization in alginate beads produced by emulsion and internal gelation. Biotechnology and bioengineering, 2011. 108(2): p. 424-434.
52. Hoesli, C. A., et al., Reversal of diabetes by βTC3 cells encapsulated in alginate beads generated by emulsion and internal gelation. Journal of Biomedical Materials Research Part B: Applied Biomaterials, 2012. 100(4): p. 1017-1028.
53. Zhang, Y. and M. Ma, Microscale Cell Encapsulation Materials and Fabrication Techniques for Type 1 Diabetes, in Microscale Technologies for Cell Engineering. 2016, Springer. p. 231-248.
54. Contreras, J. L., et al., A novel approach to xenotransplantation combining surface engineering and genetic modification of isolated adult porcine islets. Surgery, 2004. 136(3): p. 537-547.
55. Lee, D. Y., J. H. Nam, and Y. Byun, Functional and histological evaluation of transplanted pancreatic islets immunoprotected by PEGylation and cyclosporine for 1 year. Biomaterials, 2007. 28(11): p. 1957-1966.
56. Cruise, G. M., et al., A sensitivity study of the key parameters in the interfacial photopolymerization of poly (ethylene glycol) diacrylate upon porcine islets. Biotechnology and bioengineering, 1998. 57(6): p. 655-665.
57. Teramura, Y., et al., Microencapsulation of cells, including islets, within stable ultra-thin membranes of maleimide-conjugated PEG-lipid with multifunctional crosslinkers. Biomaterials, 2013. 34(11): p. 2683-2693.
58. Lin, C.-C. and K. S. Anseth, Glucagon-like peptide-1 functionalized PEG hydrogels promote survival and function of encapsulated pancreatic (3-cells. Biomacromolecules, 2009. 10(9): p. 2460-2467.
59. Kizilel, S., et al., Encapsulation of pancreatic islets within nano-thin functional polyethylene glycol coatings for enhanced insulin secretion. Tissue Engineering Part A, 2010. 16(7): p. 2217-2228.
60. Leslie-Barbick, J. E., et al., The promotion of microvasculature formation in poly (ethylene glycol) diacrylate hydrogels by an immobilized VEGF-mimetic peptide. Biomaterials, 2011. 32(25): p. 5782-5789.
61. Kin, T., et al., Xenotransplantation of pig islets in diabetic dogs with use of a microcapsule composed of agarose and polystyrene sulfonic acid mixed gel. Pancreas, 2002. 25(1): p. 94-100.
62. Gazda, L. S., et al., Encapsulation of porcine islets permits extended culture time and insulin independence in spontaneously diabetic BB rats. Cell Transplantation, 2007. 16(6): p. 609-620.
63. Leung, A., et al., Emulsion strategies in the microencapsulation of cells: pathways to thin coherent membranes. Biotechnology and bioengineering, 2005. 92(1): p. 45-53.
64. Basta, G., et al., Method for fabrication of coherent microcapsules: A new, potential immunoisolatory barrier for pancreatic islet transplantation. Diabetes, nutrition & metabolism, 1995. 8(2): p. 105-112.
65. Calafiore, R. and G. Basta, Alginate/poly-L-ornithine microcapsules for pancreatic islet cell immunoprotection, in Cell encapsulation technology and therapeutics. 1999, Springer. p. 138-150.
66. Jun, Y., et al., Microfluidics-generated pancreatic islet microfibers for enhanced immunoprotection. Biomaterials, 2013. 34(33): p. 8122-8130.
67. Tomei, A. A., et al., Device design and materials optimization of conformal coating for islets of Langerhans. Proceedings of the National Academy of Sciences, 2014. 111(29): p. 10514-10519.
68. Lee, D. Y., et al., Highly poly(ethylene) glycolylated islets improve long-term islet allograft survival without immunosuppressive medication. Tissue Eng, 2007. 13(8): p. 2133-41.
69. Wyman, J. L., et al., Immunoisolating pancreatic islets by encapsulation with selective withdrawal. Small, 2007. 3(4): p. 683-90.
70. Lee, D. Y., et al., A combination therapy of PEGylation and immunosuppressive agent for successful islet transplantation. J Control Release, 2006. 110(2): p. 290-5.
71. Jongpaiboonkit, L., W. J. King, and W. L. Murphy, Screening for 3D Environments That Support Human Mesenchymal Stem Cell Viability Using Hydrogel Arrays. Tissue Engineering. Part A, 2009. 15(2): p. 343-353.
72. Sreejalekshmi, K. G. and P. D. Nair, Biomimeticity in tissue engineering scaffolds through synthetic peptide modifications-Altering chemistry for enhanced biological response. Journal of Biomedical Materials Research Part A, 2011. 96A(2): p. 477-491.
73. Klim, J. R., et al., A defined glycosaminoglycan-binding substratum for human pluripotent stem cells. Nature methods, 2010. 7(12): p. 989-994.
74. Derda, R., et al., Defined Substrates for Human Embryonic Stem Cell Growth Identified from Surface Arrays. ACS Chemical Biology, 2007. 2(5): p. 347-355.
75. Levy-Beladev, L., et al., A family of cell-adhering peptides homologous to fibrinogen C-termini. Biochemical and Biophysical Research Communications, 2010. 401(1): p. 124-130.
76. Bellis, S. L., Advantages of RGD peptides for directing cell association with biomaterials. Biomaterials, 2011. 32(18): p. 4205-4210.
77. Hersel, U., C. Dahmen, and H. Kessler, RGD modified polymers: biomaterials for stimulated cell adhesion and beyond. Biomaterials, 2003. 24(24): p. 4385-4415.
78. Abdeen, A. A. and K. Saha, Manufacturing Cell Therapies Using Engineered Biomaterials. Trends in Biotechnology, 2017. 35(10): p. 971-982.
79. Mishra, L., R. Derynck, and B. Mishra, Transforming Growth Factor-B Signaling in Stem Cells and Cancer. Science, 2005. 310(5745): p. 68-71.
80. Watabe, T. and K. Miyazono, Roles of TGF-[beta] family signaling in stem cell renewal and differentiation. Cell Res, 2009. 19(1): p. 103-115.
81. Huang, Y.-I., et al., Effects of insulin-like growth factor-1 on the properties of mesenchymal stem cells in vitro. Journal of Zhejiang University. Science. B, 2012. 13(1): p. 20-28.
82. Zhang, J. and L. Li, BMP signaling and stem cell regulation. Developmental Biology, 2005. 284(1): p. 1-11.
83. Varga, A. C. and J. L. Wrana, The disparate role of BMP in stem cell biology. Oncogene, 0000. 24(37): p. 5713-5721.
84. Bourke, S. L. and J. Kohn, Polymers derived from the amino acid 1-tyrosine: polycarbonates, polyarylates and copolymers with poly(ethylene glycol). Advanced Drug Delivery Reviews, 2003. 55(4): p. 447-466.
85. Yu, C. and J. Kohn, Tyrosine-PEG-derived poly(ether carbonate)s as new biomaterials: Part I: synthesis and evaluation. Biomaterials, 1999. 20(3): p. 253-264.
86. Tziampazis, E., J. Kohn, and P. V. Moghe, PEG-variant biomaterials as selectively adhesive protein templates: model surfaces for controlled cell adhesion and migration. Biomaterials, 2000. 21(5): p. 511-520.
87. Baldwin, A. D. and K. L. Kiick, Reversible maleimide-thiol adducts yield glutathione-sensitive poly(ethylene glycol)-heparin hydrogels. Polymer chemistry, 2013. 4(1): p. 133-143.
88. Kong, H. J., et al., FRET measurements of cell-traction forces and nano-scale clustering of adhesion ligands varied by substrate stiffness. Proceedings of the National Academy of Sciences of the United States of America, 2005. 102(12): p. 4300-4305.
89. Kong, H. J., T. Boontheekul, and D. J. Mooney, Quantifying the relation between adhesion ligand-receptor bond formation and cell phenotype. Proceedings of the National Academy of Sciences of the United States of America, 2006. 103(49): p. 18534-18539.
90. Engler, A. J., et al., Matrix Elasticity Directs Stem Cell Lineage Specification. Cell, 2006. 126(4): p. 677-689.
91. Shiragami, N., H. Honda, and H. Unno, Anchorage-dependent animal cell culture by using a porous microcarrier. Bioprocess Engineering, 1993. 8(5): p. 295-299.
92. Dellatore, S. M., A. S. Garcia, and W. M. Miller, Mimicking Stem Cell Niches to Increase Stem Cell Expansion. Current opinion in biotechnology, 2008. 19(5): p. 534-540.
93. King, J. A. and W. M. Miller, Bioreactor Development for Stem Cell Expansion and Controlled Differentiation. Current opinion in chemical biology, 2007. 11(4): p. 394-398.
94. Sokic, S. and G. Papavasiliou, FGF-1 and proteolytically-mediated cleavage site presentation influence 3D fibroblast invasion in biomimetic PEGDA hydrogels. Acta Biomaterialia, 2012. 8(6): p. 2213-2222.
95. Loo, B.-M., et al., Binding of heparin/heparan sulfate to fibroblast growth factor receptor 4. Journal of Biological Chemistry, 2001. 276(20): p. 16868-16876.
96. Jung, D.-W., W.-H. Kim, and D. R. Williams, Reprogram or Reboot: Small Molecule Approaches for the Production of Induced Pluripotent Stem Cells and Direct Cell Reprogramming. ACS Chemical Biology, 2014. 9(1): p. 80-95.
97. Lamas, N. J., et al., Failure of Y-27632 to improve the culture of adult human adipose-derived stem cells. Stem Cells and Cloning: Advances and Applications, 2015. 8: p. 15-26.
98. Lee, W., et al., Effect of RHO-kinase inhibitor, Y27632, on porcine corneal endothelial cell culture, inflammation and immune regulation. Ocular immunology and inflammation, 2016. 24(5): p. 579-593.
99. Wu, Y., et al., ROCK inhibitor Y27632 promotes proliferation and diminishes apoptosis of marmoset induced pluripotent stem cells by suppressing expression and activity of caspase 3. Theriogenology, 2016. 85(2): p. 302-314.
100. Kurosawa, H., Application of Rho-associated protein kinase (ROCK) inhibitor to human pluripotent stem cells. Journal of Bioscience and Bioengineering, 2012. 114(6): p. 577-581.
101. Th. N. Zwietering, Suspending of solid particles in liquid by agitators, Chemical Engineering Science, Volume 8, Issues 3-4, 1958, Pages 244-253.
102. Tristan Lawson, Daniel E. Kehoe, Aletta C. Schnitzler, Peter J. Rapiejko, Kara A. Der, Kathryn Philbrick, Sandhya Punreddy, Susan Rigby, Robert Smith, Qiang Feng, Julie R. Murrell, Martha S. Rook, Process development for expansion of human mesenchymal stromal cells in a 50 L single-use stirred tank bioreactor, In Biochemical Engineering Journal, Volume 120, 2017, Pages 49-62.
103. Quinn, J. F., M. R. Whittaker, and T. P. Davis, Glutathione responsive polymers and their application in drug delivery systems. Polymer Chemistry, 2017. 8(1): p. 97-126.
104. Yang, F., et al., Bone regeneration using cell-mediated responsive degradable PEG-based scaffolds incorporating with rhBMP-2. Biomaterials, 2013. 34(5): p. 1514-1528.
105. Era, J., et al., Storage stability of biodegradable polyethylene glycol microspheres. Materials Research Express, 2017. 4(10): p. 105403.
106. Zustiak, S. P., et al., Hydrolytically Degradable Poly(Ethylene Glycol) Hydrogel Scaffolds as a Cell Delivery Vehicle: Characterization of PC12 Cell Response. Biotechnology progress, 2013. 29(5): p. 1255-1264.
107. Zustiak, S. P. and J. B. Leach, Hydrolytically degradable poly(ethylene glycol) hydrogel scaffolds with tunable degradation and mechanical properties. Biomacromolecules, 2010. 11(5): p. 1348-1357.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A capsule for growing or storing cells, comprising:
a shell defining an interior compartment; and
a substrate for cell attachment located within the interior compartment,
wherein the substrate comprises a polymer and one or more adhesion molecules.

2. The capsule of claim 1, wherein the substrate for cell attachment is an inner surface of the shell.

3. The capsule of claim 1, wherein the substrate for cell attachment is a hydrogel disposed within the interior compartment.

4. The capsule of claim 3, wherein the hydrogel comprises cross-linked polyethylene glycol (PEG), cross-linked polyethylene glycol diacrylate (PEGDA), or a combination thereof.

5. The capsule of any one of claims 1-4, wherein the shell comprises a polymer selected from the group consisting of polyethylene glycol (PEG), polyethylene glycol diacrylate (PEGDA), alginate, chitosan, PEG copolymerized with alginate or chitosan, PEGDA copolymerized with alginate or chitosan, poly(lactic-co-glycolic acid) (PLGA), poly-L-lysine (PLL), polydimethylsiloxane (PDMS), and polyacrylamide, polyacrylamide, poly(N-isopropylacrylamide) (PNIPAAm), poly[2-(methyacryloxy)ethyl phosphorylcholine]-block-(glycerol monomethacrylate) (PMPC-PGMA), x-acetylene-poly(tert-butyl acrylate) (PtBA), poly(vinyl alcohol) (PVA), poly(acrylic acid) (PAA), poly(divinylbenzene-co-glycidylamethacrylate) (P(DVB-GMA)), poly(amidoamine) (PAMAM), poly(D-glucosamidoethylenemethacrylate) (PGAMA), poly(2-lactobionamido ethylmethacrylate (PLAMA), alkyl thioether end-functionalized poly(methacrylic acid) (PMAA-DDT), poly(ethylene glycol)-phosphine (PEG-phosphine), poly(vinyl pyrollidone) (PVP), poly(acrylic acid)-octylamine (PAA-octylamine), and poly(maleic anhydride-alt-1-octadecene-block-poly(ethylene glycol) (PMAO-PEG), or a combination thereof.

6. The capsule of any one of claims 1-5, wherein the one or more adhesion molecules include an adhesion peptide.

7. The capsule of claim 6, wherein the adhesion peptide is selected from the group consisting of RGDS, YIGSR, IKVAV, REDV, GKKQRFRHRNRKG, RNIAEIIKDI, and KTRWYSMKKTTMKIIPFNR, or any combination thereof.

8. The capsule of any one of claims 1-5, wherein the one or more adhesion molecules include an adhesion protein.

9. The capsule of claim 8, wherein the adhesion protein is selected from the group consisting of collagen type I, fibronectin, laminin, denatured collagen, collagen type IV, and Matrigel®, or any combination thereof.

10. The capsule of any one of claims 1-5, wherein the one or more adhesion molecules includes an antibody.

11. The capsule of claim 10, wherein the antibody is an antibody that binds a cell surface receptor.

12. The capsule of claim 10, wherein the antibody is an antibody that binds at least one of CD3, CD28, and CD40.

13. The capsule of any one of claims 1-5, wherein the one or more adhesion molecules includes a nonpeptide small molecule.

14. The capsule of claim 13, wherein the nonpeptide small molecule is a non-steroidal anti-inflammatory molecule.

15. The capsule of claim 13, wherein the small molecule is stemregulin, reversine, or a combination thereof.

16. The capsule of any one of claims 1-5, wherein the one or more adhesion molecules are molecules that affect viability, proliferation, survival, growth, differentiation, or any combination thereof.

17. The capsule of any one of claims 1-16, wherein the substrate further comprises a growth factor.

18. The capsule of claim 17, wherein the growth factor is conjugated to the polymer.

19. The capsule of claim 1 or 2, wherein the interior compartment includes a liquid comprising a growth factor.

20. The capsule of any one of claims 16-19, wherein the growth factor is selected from the group consisting of FGF, TGF-β1, VEGF, PDGF-BB, PDGF, IGF1, stem cell factor (SCF), thrombopoeitin (TPO), FMS-like tyrosine kinase 3 ligand (Flt-3L), erthryopoeitin, DL-1 notch ligand, Wnt, stromal derived factor (SDF)-1, interleukin (IL)-2, IL-3, IL-4, IL-6, IL-7, IL-15, IL-15R, CD40L, G-CSF, GM-CSF, 4-1BB and a BMP superfamily member, or any combination thereof.

21. The capsule of any one of claims 1-20, wherein the shell is porous.

22. The capsule of claim 21, wherein the porous shell has a pore size of about 10 nm to about 35 nm.

23. The capsule of claim 22, wherein the pore size is about 20 nm.

24. The capsule of any one of claims 1-23, wherein the shell further comprises an enzyme-sensitive peptide.

25. The capsule of claim 24, wherein the enzyme-sensitive peptide is a protease-sensitive peptide.

26. The capsule of claim 24, wherein the peptide is sensitive to an enzyme selected from the group consisting of trypsin, collagenase, DNase, RNase, and horseradish peroxidase, or any combination thereof.

27. The capsule of any one of claims 1-26, further comprising a cell adhered to the substrate.

28. The capsule of claim 27, wherein the adherent cell is a selected from the group consisting of a stem cell, a pancreatic islet, a peripheral blood mononuclear cell, an endothelial progenitor cell, a blood fibrocyte, a bone marrow cell, a T cell, a B cell, a dendritic cell, an NK cell, a monocyte, a CD34+ cell, a hepatocyte, a gastrointestinal cell, a skin cell, a skin progenitor cell, a cancer cell, a hybridoma cell, a prokaryotic cell, a HEK293T packaging cell, a yeast cell, and a pancreatic precursor cell.

29. The capsule of claim 27, wherein the cell is selected from the group consisting of a Mesenchymal Stem Cell (MSCs), a Chinese Hamster Ovary (CHO) cell, a Madin-Darby Canine Kidney Epithelial (MDCK) cell, and a Vero cell, a neural stem cell, an embryonic stem cell, and a pluripotent stem cell.

30. The capsule of any one of claims 1-29, further comprising of a DNA-containing molecule or an RNA-containing molecule.

31. The capsule of claim 30, wherein the DNA-containing molecule or RNA-containing molecule is carried by a viral particle adhered to the substrate.

32. The capsule of claim 30, wherein the DNA-containing molecule or RNA-containing molecule contains at least one of cDNA, plasmid DNA, transposable DNA, a viral vector, modified RNA, siRNA, miRNA, an antisense oligonucleotide, a gene editing molecule, a zinc finger nuclease, and a meganuclease.

33. The capsule of claim 32 wherein the DNA-containing molecule or RNA-containing molecule is contained within a lipid vesicle.

34. A method of storing cells, comprising:
encapsulating cells in a capsule of claim 1, wherein the cells adhere to the substrate within the interior compartment of the capsule; and
maintaining the cells within the capsule in a viable state.

35. The method of claim 34, wherein the capsule comprises a porous shell, and further wherein encapsulating the cells includes exposing the porous shell of the capsule to the cells, the cells translocating into the interior compartment of the shell.

36. The method of claim 34 or 35, wherein the capsule includes a culture medium in the interior compartment, thereby producing a suspension culture of encapsulated cells, and the method further comprises growing or expanding the cells in the suspension culture.

37. The method of claim 36, wherein the suspension culture is a stirred-tank suspension culture.

38. The method of any one of claims 34-37, further comprising degrading the shell of the capsule and harvesting the cells.

39. A cell culture kit, comprising:
a first composition comprising a polymer precursor material and an adhesion molecule; and
a second composition comprising reagents for polymerizing the polymer precursor material to form a capsule having a shell that comprises a polymer produced by polymerization of the precursor material and the adhesion molecule, wherein the adhesion molecule is present on an inner surface of the shell.

40. The cell culture kit of claim 39, wherein the first composition further comprises a growth factor.

41. The cell culture kit of claim 39 or claim 40, wherein the first composition further comprises an enzyme-sensitive peptide.

42. The cell culture kit of any one of claims 39-41, wherein the second composition further comprises a cross-linking reagent for forming a hydrogel within the interior compartment of the shell.

43. The cell culture kit of any one of claims 39-42, wherein the first composition is lyophilized.

44. A system for producing capsules:
a first solution comprising a polymer and a hydrophilic photoinitiator;
a second solution comprising an emulsion including a hydrophobic photoinitiator,
at least one pump configured to mix the first solution and the second solution and configured to pump the mixed first and second solutions through a light-reflecting apparatus; and
a light emitting device configured to illuminate the light-reflecting apparatus.

45. The system of claim 42, further comprising a collection vessel, wherein the mixed first and second solutions are pumped continuously through the light-reflecting apparatus to the collection vessel.


SEQUENCE LISTING (11 sequences)

SEQ ID NO: 1 - protein, 4 aa, ARTIFICIAL SEQUENCE
  cell adhesion peptide
RGDS

SEQ ID NO: 2 - protein, 5 aa, ARTIFICIAL SEQUENCE
  cell adhesion peptide
YIGSR

SEQ ID NO: 3 - protein, 5 aa, ARTIFICIAL SEQUENCE
  cell adhesion peptide
IKVAV

SEQ ID NO: 4 - protein, 4 aa, ARTIFICIAL SEQUENCE
  cell adhesion peptide
REDV

SEQ ID NO: 5 - protein, 13 aa, ARTIFICIAL SEQUENCE
  cell adhesion peptide
GKKQRFRHRNRKG

SEQ ID NO: 6 - protein, 10 aa, ARTIFICIAL SEQUENCE
  cell adhesion peptide
RNIAEIIKDI

SEQ ID NO: 7 - protein, 19 aa, ARTIFICIAL SEQUENCE
  cell adhesion peptide
KTRWYSMKKTTMKIIPFNR

SEQ ID NO: 8 - protein, 12 aa, ARTIFICIAL SEQUENCE
  protease sensitive peptide
GGGPQGIWGQGK

SEQ ID NO: 9 - protein, 10 aa, ARTIFICIAL SEQUENCE
  protease sensitive peptide
GGGLGPAGGK

SEQ ID NO: 10 - protein, 211 aa, human immunodeficiency virus
IRVENITNNAKTIIVSLNKSVNIICTRPNNNTRKSIRIGPGQAFYATGDIIGDIREAHCNISKKEWNETLYMVSKKLREHFPNKTIEFAPPSGGDLEVTTHSFNCGGEFFYCNTSALFNRTYRSNDTESNMSNDTESNNSTITLKCRIKQIINMWQEVGRAMYAPPIEGIIKCNSNITGLLLTRDGGYNNKTNISEIFRPGGGNMRDNWRS

SEQ ID NO: 11 - protein, 9 aa, ARTIFICIAL SEQUENCE
  protease sensitive peptide
GGLGPAGGK